(19) United States
(12) Patent Application Publication
(10) Pub. No.: US 2013/0028956 A1
(43) Pub. Date: Jan. 31, 2013

(54) METHOD FOR PREVENTING OR TREATING MEMORY IMPAIRMENT AND PHARMACEUTICAL COMPOSITIONS USEFUL THEREFORE

(71) Applicants: Andre Fischer, Goettingen (DE); Athanasios Zovoilis, Chalkida (GR); Hope Agbemenyah, Goettingen (DE)

(72) Inventors: Andre Fischer, Goettingen (DE); Athanasios Zovoilis, Chalkida (GR); Hope Agbemenyah, Goettingen (DE)

(21) Appl. No.: 13/193,841

(22) Filed: Jul. 29, 2011

(51) Int. Cl. A61K 31/711; A61K 38/16; A61K 38/43; A61K 39/395; C12Q 1/68; A61K 39/44; A61K 9/14; A61K 9/127; A61P 25/28; A61K 31/74; A61K 31/713

(52) U.S. Cl. 424/450; 514/44 R; 424/78.08; 514/17.7; 424/94.1; 424/130.1; 514/44 A; 424/178.1; 424/497; 435/6.11

(57) ABSTRACT

In a first aspect, the present invention relates to a method for prevention or treating memory impairment and/or a neurodegenerative condition, disorder or disease in a subject in need thereof, comprising administering to the subject a therapeutically effective amount of a compound targeting a miRNA or any precursor, or targeting the activity of said miRNA or any precursor thereof. In particular, the present invention relates to a method wherein the targeted miRNA is miR-34. Moreover, the present invention relates to a method for improving memory functionality in a subject suffering from memory loss, said method comprises the reduction or reducing the level of miR-34 levels or precursor molecule levels in brain tissue of said subject administering to the subject a therapeutically effective amount of a compound targeting miR-34 or any precursor thereof.

Moreover, the present invention relates to a method of inducing memory loss in an individual comprising administering to said individual an effective amount of one or more compounds having the functionality of miR-34, in particular of miR-34c or any precursor thereof. In addition, a method for diagnosing or predicting memory impairment and/or neurodegenerative condition, disorder or disease is provided. Finally, the present invention relates to a pharmaceutical composition containing a compound targeting miR-34 or precursor molecules thereof.

### BACKGROUND OF THE INVENTION

In a first aspect, the present invention relates to a method for prevention or treating memory impairment and/or a neurodegenerative condition, disorder or disease in a subject in need thereof, comprising administering to the subject a therapeutically effective amount of a compound targeting a miRNA or any precursor, or targeting the activity of said miRNA or any precursor thereof. In particular, the present invention relates to a method wherein the targeted miRNA is miR-34. Moreover, the present invention relates to a method for improving memory functionality in a subject suffering from memory loss, said method comprises the reduction or reducing the level of miR-34 levels or precursor molecule levels in brain tissue of said subject administering to the subject a therapeutically effective amount of a compound targeting miR-34 or any precursor thereof.

Moreover, the present invention relates to a method of inducing memory loss in an individual comprising administering to said individual an effective amount of one or more compounds having the functionality of miR-34, in particular of miR-34c or any precursor thereof. In addition, a method for diagnosing or predicting memory impairment and/or neurodegenerative condition, disorder or disease is provided. Finally, the present invention relates to a pharmaceutical composition containing a compound targeting miR-34 or precursor molecules thereof.

### PRIOR ART

Age associated dementias consist as a serious health and social problem for our societies, with their prevalence increasing as lifespan expands. The hippocampus is a key brain structure for memory formation and amongst the first brain regions to be affected during aging and in neurodegenerative dementias such as Alzheimer's disease (AD), which arises on the pathological background of amyloid β plaques, neurofibrilary tangels, synaptic and neuronal loss. The major hallmark of AD is however severe memory impairment eventually leading to dementia. Efficient therapeutic strategies to treat memory decline are still missing, which is in part due to the fact that mechanisms underlying the pathogenesis of memory impairment in AD are only partially understood. Deregulated hippocampal gene-expression has been observed during aging and brain diseases of animal models as well as in human patients, e.g. Peleg et al, 2010; Science 328 (753): 753-756. To understand the mechanisms that are causative for this de-regulated transcriptome plasticity is of utmost importance and may lead to the development of novel therapeutic strategies.

A possible regulatory mechanism that is only beginning to emerge is the regulation of gene-expression via microRNAs (miRNAs). miRNAs play key roles in a wide range of biological processes and regulate gene expression mainly at the translational level. Recent work implicated miRNAs with neuronal function e.g. Konopka et al, 2009; 3 Neurosci 30(44): 14835-14842 and the pathogenesis of AD e.g. Hébert et al, 2008; Proc Natl Acad Sci USA 205(17): 6415-6420. Until now, hippocampal miRNAs have been investigated using targeted or high throughput approaches, but the latter have been limited to either microarrays or a relative small number of sequenced clone reads. Moreover, microarray analysis is prone to cross-hybridization between similar sequences, an attribute very common in case of miRNAs. In addition, competitive hybridization is a relative measure, limiting the dynamic range of high confidence data. Massive parallel sequencing of small RNAs offers remedies to the above problems and can provide an unprecedented insight into the small non coding transcriptome of the cells e.g. described by Hurd & Nelson, 2009; Brief Funct Genomic Proteomic 8(12): 174-183.

MicroRNAs (miRNas or miR) are a class of single stranded non coding RNAs (ncRNAs) that have been conserved in evolution from plants to animals. Mature miRNAs are typically around 17 to 24 nucleotides in length, but may be longer or shorter. They are generated in cells from miRNA precursors as a result of a series of RNA processing steps. First a miRNA precursor transcript having a hairpin structure is produced. The mature miRNA is located within one arm/strand of this precursor hairpin (the opposite strand of the hairpin, known as the star (*) strand, is generally degraded. The miRNA precursor is processed in the nucleus to form a pre-miRNA which is exported to the cytoplasm. The pre-miRNA undergoes further processing in the cytoplasm to form the mature miRNA. It is this mature miRNA that inhibits the expression of its target gene at the post transcriptional level by binding to the miRNA of the target gene by Watson-Crick base pairing. miRNAs have been found to have roles in a variety of biological processes including developmental timing, differentiation, apoptosis, cell proliferation, organ development, and metabolism. miRNA can be divided into a seed region nucleotide sequence and a non-seed region nucleotide sequence. Said seed region typically consists of five to seven nucleotides or more representing the site of hybridization with the target nucleotide sequence. That is, a microRNA family refers to a group of microRNAs species that share identity across at least five or sixth consecutive nucleotides within nucleotide positions 1 to 12 of the 5′ end of the microRNA molecule, also referred to as the “seed region”, see Brennecke, J. et al., PloS Biol. 3(3): P85 (2005).

Usefulness of miRNA as therapeutic agents for treating various diseases are described in the art, e.g. WO 2009/126650 discloses the use of mi-126 and inhibitors of mi-126 for modulating angiogenesis. WO 2008/137867 describes compositions comprising miR-34 therapeutic agents for treating cancer. Cogswell J. P, et al., J. Alzheimers disease, 14, p 27-41, 2008, describes the identification of miRNA changes in Alzheimer's disease brain and CSF yields putative biomarkers and insights into disease pathways. It is outlined therein, that miRNA expression is altered in Alzheimer's CSF. In addition, it is shown therein that changes for miRNA in CSF of normal and Alzheimer's disease can be detected. WO 2008/154333 relates to methods and compositions for identifying genes or genetic pathways modulated by miR-34, using miR-34 to modulate a gene or gene pathway, as well as of using this profile in assessing the condition of a patient and/or treating the patient with an appropriate miRNA.

Thus, it is generally accepted that miRNA may be useful for treatment or prevention of various diseases, disorders and conditions.

Herein, the present inventors recognized that miRNA impacts memory and pathogenesis of brain diseases. That is, the present inventors recognized that memory impairment and/or a neurodegenerative condition, disease or disorder may be prevented or treated in a subject in need thereof comprising administering to said subject a therapeutically effective amount of a compound targeting a miRNA or any precursor, or targeting the activity of said miRNA or any precursor thereof.

### SUMMARY

Hence, in a first aspect, the present invention relates to a method for preventing or treating memory impairment and/or a neurodegenerative condition, disease or disorder in a subject in need thereof, comprising administering to the subject a therapeutically effective amount of a compound targeting a miRNA or any precursor, or targeting the activity of said miRNA or any precursor thereof.

It has been recognized by the present inventors that miR is implicated with the pathogenesis of cognitive decline. For example, it has been found that miR-34c represents a negative constraint of memory consolidation and, in addition, miR-34 levels are elevated in the hippocampus of AD patients. Hence, said miR represents a marker for the onset of cognitive disturbance linked to Alzheimer's disease and, thus, targeting said miR represents a suitable therapy in memory impairment and/or degenerative condition, disease or disorder. Moreover, it has been recognized that administration of said miR molecules induces memory loss in an individual.

Thus, in a further aspect, the present invention relates to a method of inducing memory loss in an individual comprising administering to said individual an effective amount of one or more compounds having the functionality of the small-noncoding miR-34, in particular miR-34c or any precursor thereof or a miRNA sharing the same miRNA seed with miR-34, in particular, administering miRNA-34c or any precursor thereof.

Moreover, the present invention relates to a method for improving memory functionality in a subject suffering from memory loss, the method comprising reducing the level of miR-34 levels or precursor molecule levels in brain tissue of the subject by administering to the subject a therapeutic effective amount of a compound targeting miR-34 or any precursor thereof.

In addition, the present invention provides a method for diagnosing or predicting memory impairment and/or a neurodegenerative condition, disease or disorder in a subject comprising the steps of
determining the level of expression of miR-34 or any precursor thereof, in particular, miR-34c in a biological sample, like in tissue, blood, liquor or cerebrospinal fluid sample
comparing the level of expression of miR-34 or any precursor thereof, in particular, miR-34c, with a reference sample of a subject not afflicted with or suspected to have memory impairment and/or a neurodegenerative condition, disease or disorder;
predicting or diagnosing memory impairment and/or a neurodegenerative condition, disease or disorder based on the expression level of miR-34 or any precursor thereof, in particular, based on miR-34c expression level in the biological sample, like in tissue, blood, liquor or cerebrospinal fluid.

Finally, the present invention relates in another aspect to a pharmaceutical composition containing a compound targeting miR-34, pre-miR-34 precursor, pri-miR-34 precursor, or a miRNA sharing the same miRNA seed with miR-34, in particular, targeting miR-34c or any precursor thereof together with a pharmaceutically acceptable carrier, diluent or recipient.

### BRIEF DESCRIPTION OF THE DRAWINGS

FIG. 1. Massive parallel sequencing of small RNA libraries reveals the hippocampal miRNAome (a) Proportion of detected mature miRNAs, regarding the total number of known genes and miRNAs in miRBase. (b) Proportion of sequence counts per miRNA with respect to the total number of counts attributed to miRNAs in hippocampus. (c) Sequence counts of top ranking hippocampal miRNAs relative to the respective counts in whole brain −(log 2 scale).

FIG. 2. miR-34c targets learning associated genes. Enrichment of learning associated genes up-regulated 60 min after fear conditioning in seeds of miRNAs highly expressed in hippocampus.

FIG. 3. High levels of miR-34c in mouse models for learning impairment (a) Hippocampal miR-34c expression levels determined by qPCR in 2 mouse models for memory impairment, (left) 24-month old mice (n=4; p=0.02), (right) 12 m old APPPS1-21 mice (n=7.6; p=0.01). (b) Left panel: 3 and 24-month old C57B16J mice were subjected to contextual fear conditioning and freezing behavior was analyzed 24 h later during the memory test. Freezing was significantly reduced in 24-month old mice (n=8/group, *P<0.05). Right panel: Twelve-month-old male APPPS1-21 mice were exposed to contextual fear conditioning. When compared to age-matched control littermates associative memory was impaired in APPPS1-21 mice. No difference in explorative behavior or response to the foot-shock was observed amongst groups (data not shown). (c) SIRT1 protein levels are significantly reduced in the hippocampus of aged (p=0.02 vs young, n=4) and APPPS1-21 mice (p=0.01 vs wild type, n=7.6). In line with a translational repression mode of action of miR-34c on SIRT1, Sirt1 mRNA levels were not affected. Error bars indicate SEM.

FIG. 4 a-c. miR-34c is up-regulated in AD patients. qPCR analysis was used to measure miR-34c levels in postmortem tissue samples from AD patients and age-matched controls. (a) miR-34c levels (qPCR) are elevated in the hippocampus (p=0.05) of human AD patients (n=6; 4 females, 2 males) when compared to age-matched controls (n=8, 2 females, 6 males). Five AD cases were diagnosed as Braack & Braack stages VI and one was diagnosed Braack & Braack V. There was no significant difference for the age (b) or post-mortem delay (c) amongst groups. Error bars indicate SEM.

FIGS. 4 d+e. miR-34c is regulated during fear conditioning and its expression correlates with SIRT1 levels. Sirt1 is a confirmed target of the miR34 family. Sirt1 is expressed in the hippocampus and essential for memory function. Sirt-1 mRNA incorporates in its 3′UTR the miRNA seed for miR-34c and related miRNAs. (d) Cortical neurons (DIV5+3) were co-transfected with the dual luciferase 3′UTR reporters and miRNA expression constructs. The 3′UTR of renilla contained two artificial miR-34 target sites or SIRT1 3′UTR (wild type or a mutant lacking both putative miR-34 seed regions). Relative expression was determined by normalizing the ratio of renilla and firefly luciferase activity to the effect of each miRNA on a control renilla-luciferase reporter (and miR-143 expressing vector). Statistical analysis using two-way ANOVA comparing miR-34c and miR-143 and SIRT1 wildtype and mutant (**** denotes p<0.001, n=12). (e) mRNA and protein levels of SIRT1 60 min, 180 min and 24 h after FC. Note, that the reduction in SIRT1 levels at 180 min corresponds to increase of miR-34c levels at that time point (n=4; *P<0.05).

FIG. 5. High levels of miR-34c are implicated with memory impairment (a) left panel: Experimental design for intrahippocampal administration of miR-34c mimic and the respective scramble miR. Right panel: Elevated hippocampal miR-34c levels after miR-34c mimic injection (n=5.7; p=0.05) (b) Upper panel, experimental design. Impaired learning in 3-month-old wild type mice with high levels of miR-34c after treatment with the miRNA mimic (miR-34c) (n=6, p<0.001). (c) Hippocampal SIRT1 levels correlate with miR-34c expression. miR-34c was injected into the hippocampus as shown in FIG. 4a (n=4; p=0.04). Sirt1 mRNA and protein levels were measured 24 h after fear conditioning. While elevation of miR-34c did not significantly affected Sirt1 mRNA levels (there was even a trend for increased Sirt1 mRNA, possible through compensatory mechanisms) the present inventors observed a dramatic effect in translation of this mRNA, leading to a significant repression of Sirt-1 protein levels. Error bars indicate SEM. (d) Relative SIRT1 protein/mRNA ratio for the experiment shown in (a).

FIG. 6. miR-34c induces reduction of SIRT1 protein levels via its binding to SIRT1 3′UTR (a) Experimental design. Upper panel: The present inventors designed miR-34c-Sirt1 protectors for both miR-34c binding sites within the 3′ UTR of the Sirt1 mRNA. These protectors prevent miR34c mimic from binding to the Sirt1 mRNA. Lower panel: A control target protector oligonucleotide was used to exclude non-specific effects (b) Upper panel: Experimental design. Lower panel. While intrahippocampal injection of miR-34c mimic along with the target protector negative control impaired associative learning in the contextual fear conditioning paradigm, co-injection with the miR-34c-Sirt1 protector rescues learning impairment (n=8.8; p=0.04). Mice co-injection with of a scramble miR and the target protector negative control served as an additional control (n=8.6; p=0.04). (c) SIRT1 protein (left) and in RNA levels (right) in mice treated with the miR-34c mimic and miR-34c-Sirt1 protector compared to mice injected with miR-34c mimic along with the target protector negative control (p=0.07, n=7.5). Mmu, mus musculus. Error bars indicate SEM.

FIG. 7. Targeting miR-34c seed rescues learning impairment in mouse model for AD (a) Experimental design. (b) Impaired learning of 12 in APPPS1-21 mice (n=8.7; p=0.001) is partially rescued after inhibition of the miR-34c activity (n=7.8; p=0.04). (c) SIRT1 protein (left) and mRNA levels (left) in miR34 seed inhibitor treated mice (*P<0.05; n=8). Error bars indicate SEM.

FIG. 8. Targeting miR-34c seed enhances learning in 20-month-old wild type animals. (a) Experimental design. (b) Inhibiting miR-34c activity via intrahippocampal injection of miR34 seed inhibitor enhances associative memory as measured in the contextual fear conditioning paradigm in 20 month old animals (n=8.7 P=0.02). (c) miR-34 seed inhibitor treated 20-month-old mice show elevated SIRT1 protein levels (P=0.03) while Sirt1 mRNA levels were unaffected (n=7 p3=0.04). FC; contextual fear conditioning. Error bars indicate SEM.

FIG. 9. Targeting miR-34c seed enhances learning in 3-month-old wild type animals. (a) Experimental design. (b) Inhibiting miR-34c activity in 3-month-old wild type mice via intrahippocampal injection of miR34 seed inhibitor enhances associative learning as measured in the contextual fear conditioning paradigm (n=5.6 p=0.01), (c) In contrast to the effect observed in disease models were SIRT1 protein is down-regulated inhibiting miR-34c activity does not have significant effects on SIRT1 protein in 3-month old mice, although there was a non-significant trend (n=6). This data suggests that the effect of miR34c on memory formation in young wild type mice involves targets. FC, contextual fear conditioning. Error bars indicate SEM.

### DETAILED DESCRIPTION OF THE PRESENT INVENTION

The present invention provides a method for preventing or treating memory impairment and/or a neurodegenerative condition, disease or disorder in a subject in need thereof, comprising administering to the subject a therapeutically effective amount of a compound targeting a miRNA or any precursor, or targeting the activity of said miRNA or any precursor thereof.

As used herein, the words “comprising”, “having”, “including” or “containing” are inclusive and open-ended and do not exclude additional, unrecited elements or method steps. Said terms include the specific embodiment of consisting.

“microRNA”, “miRNA”, or “miR” refers to small, non-protein coding RNA molecules that are expressed in a diverse array of eukaryotes, including mammals. While mature fully processed miRNAs are typically about 15 to 30, 15 to 25 or 20 to 30 nucleotides in lengths, and more often between 16 to 24, 17 to 23, 18 to 22, 19 to 21, or 21 or 24 nucleotides in lengths, microRNAs include processed sequences as well as corresponding long primary transcripts (pri-mRNAs) and processed precursors (pre-miRNAS).

As used herein, “targeting of miRNA” refers to administering or providing “an effective amount” or “therapeutically effective amount” in an amount sufficient to produce the desired effect, e.g. inhibition of miR activity or interference with miR and the target site or binding site of miR.

As used herein, the term “complementary” refers to nucleic acid sequences that are capable of base-pair according to the standard of Watson-Crick complementary rules. That is, the larger purines will base pair with a smaller pyrimidines to form combination of guanine paired with cytosine (G:C) and adenine paired with either thymine (H:T) in the case of DNA, or adenine paired with uracil (H:U) in the case of RNA. Complementarity is expressed as percentage sequence complementarity.

As used herein, the term “miR-34 family” refers to miR-34a, miR-34b, miR-34c, and miR-449 a, b, and c and miR-699.

As used herein, the term “miR-34” refers to one or more of miR-34a, miR-34b and miR-34c.

As used herein, the term “mir-34” seed region refers to Seq. ID No. 72 (GGCAGUGU).

As used herein, an “isolated nucleic acid” is a nucleic acid molecule that exists in a physical form that is non identical to any nucleic acid molecule of identical sequence as formed in nature; “isolated” does not require although it does not prohibit, that the nucleic acid as described has itself been physically removed from its native environment. For example, a nucleic acid can be said to be “isolated” when it includes nucleotides and/or nucleotide bonds not found in nature. When instead composed of natural nucleotides in phosphodiester linkage, a nucleic acid can be set to be “isolated” when it exist at a purity not found in nature, where purity can be adjusted with respect to the presence of nucleic acids of other sequences, with respect of the present of the proteins, with respect to the presence of lipids etc. As notified, “isolate nucleic acid” includes nucleic acids integrated in the host cell chromosome at a heterologous site, recombinant fusions of a native fragment to a heterologous sequence, recombinant factors present as episomes or as integrated into a host cell chromosome.

As used herein “specific binding” refers to the ability of two molecular species can currently present in a heterogeneous (inhomogeneous) sample to bind to one another in preference to binding to other molecule species in a sample. Typically, a specific binding interaction will discriminates over adventicious binding interactions in the reaction by at least two fold, more typically by at least 10 fold, often at least 100 fold.

As used herein “subject” refers to an organism also termed individual. Said organism may be an animal including mammals, typically human.

In a preferred embodiment of the present invention, the method relates to administering to the subject a therapeutically effective amount of a compound targeting miR-34 or any precursor thereof, or a miR showing the same miR seed with miR-34 or any precursor thereof. In this connection, the miR seed of the miR-34 family is: GGCAGUGU (Seq. ID No. 72). Thus, in a preferred embodiment, the compound targeting miR-34 is a compound comprising the complementary sequence to the miR-34 family seed.

In a particularly preferred embodiment, the targeted miRNA is miR-34c or any precursor thereof.

The compound which may be administered according to the present invention is preferably an oligonucleotide in particular, a RNA or DNA nucleotide, which may be modified, or an artificially synthesized polymer able to interfere between the miRNA as identified herein and the target, in particular, the miRNA seed, like the miR-34 seed as described herein and/or to reduce the expression of said miRNA or antagonise the activity of said miRNA. In this connection, the term “oligonucleotide” refers to compounds based on nucleotides having a size typically in between 6 to 50 nucleic acids, like 8 to 40, for example, a nucleotide sequence of at least 10, 11, 12, 13, 14, 15, 16, 17, 18, 19, 20, 21, 22 nucleotides or molecules having at most 50, 49, 48, 47, 46, 45, 44, 43, 42, 41, 40, 39, 38, 37, 36, 35, 34, 33, 32, 31, 30 nucleotides.

A “miR-34 nucleic acid sequence” includes the full length precursor of miR-34, or complement thereof or processed (i.e., mature) sequence of miR-34 and related sequences set forth herein, as well as 5, 6, 7, 8, 9, 10, 11, 12, 13, 14, 15, 16, 17, 18, 19, 20, 21, 22, 23, 24, 25, 26, 27, 28, 29 or more nucleotides of a precursor miRNA or its processed sequence, or complement thereof, including all ranges and integers there between. In certain embodiments, the miR-34 nucleic acid sequence contains the full-length processed miRNA sequence or complement thereof and is referred to as the “miR-34 full-length processed nucleic acid sequence”. In still further aspects, the miR-34 nucleic acid comprises at least one 5, 6, 7, 8, 9, 10, 11, 12, 13, 14, 15, 16, 17, 18, 19, 20, 21, 22, 23, 24, 25, 50 nucleotide (including all ranges and integers there between) segment or complementary segment of a miR-34 that is at least 75, 80, 85, 90, 95, 98, 99 or 100% identical to SEQ ID NOs: 1 to 72. The general term miR-34 includes all members of the miR-34 family that share at least part of a mature miR-34 sequence.

A “miR-34 nucleic acid sequence” includes all or a segment of the full length precursor of miR-34 family members.

In some embodiments, the present invention is directed to miR-34 and to nucleotides having or comprising SEQ ID NOs. 1 to 71 and homologues thereof. In addition, the present invention is directed to fragments and variants of SEQ IDs:1 to 71 that demonstrate the same effect as the nucleic acids of Seq. IDs 1 to 71. Accordingly, the present invention relates to fragments and variants of SEQ ID NOs 1 to 71 having greater than about 70%, or greater than about 75%, or greater than about 80%, or greater than about 85%, or greater than about 90%, or greater than about 95%, or greater than about 99% of their nucleotides identical to those of SEQ IDs 1 to 71, and that bind to the miR-34 target gene(s) and modulate postnatal tissue repair. In addition, the present invention also provides fragments and variants of SEQ IDs 1 to 71 that differ from SEQ IDs 1 to 71 by a certain number of nucleotides. For example, in one embodiment, the present invention provides sequences that differ from SEQ ID NOs: 1 to 71 by no more than 10 nucleotides, or no more than 9 nucleotides, or no more than 8 nucleotides, or no more than 7 nucleotides, or no more than 6 nucleotides, or no more than 5 nucleotides, or no more than 4 nucleotides, or no more than 3 nucleotides, or no more than 2 nucleotides, or no more than 1 nucleotide, and that bind to the miR-34 target gene(s) and modulate postnatal tissue repair.

It is well known in the art that while in deoxyribonucleic acids the complementary nucleotide to Adenosine (“A”) is thymidine (“T”), in ribonucleic acids the complementary nucleotide to A is uracil (“U”). Thus, the nucleotide T in a deoxyribonucleic acid is the equivalent of the nucleotide U in a ribonucleic acid, and vice versa.

For example, the nucleic acid-based miR-34 sequences of the present invention may comprise ribonucleotides, deoxyribonucleotides, 2′-modified nucleotides, phosphorothioate-linked deoxyribonucleotides, peptide nucleic acids (PNAs), locked nucleic acids (LNAs), ethylene nucleic acids (ENA), certain nucleobase modifications such as 2-amino-A, 2-thio (e.g., 2-thio-U), G-clamp modifications, antagomirs, morpholinos, nucleic acid aptamers, or any other type of modified nucleotide or nucleotide derivative that is capable of Watson-Crick type base pairing with an miRNA. For example, in addition to naturally occurring DNA and/or RNA nucleotide bases, non-naturally occurring modified nucleotide bases that can be used in the miR-34 sequences of the invention include, but are not limited torn 8-oxo-guanine, 6-mercaptoguanine, 4-acetylcytidine, 5-(carboxyhydroxyethyl) uridine, 2′-β-methylcytidine, 5-carboxymethylamino-methyl-2-thioridine, 5-carb Ipseudouridine, beta-D-galactosylqueosine, 2′-Omethylguanosine, inosine, N.sup.6-isopentenyladenosine, 1-methyladenosine, 1-methylpseudouridine, 1-methylguanosine, 1-methylaminomethyllinosine, 2,2-dimethylguanosine, 2-methyladenosine, 2-methylguanosine, 3-methylcytidine, 5-methylcytidine, N.sup.6-methyladenosine, 7-methylguanosine, 5-methylaminomethyluridine, 5-methoxyaminomethyl-2-thiouridine, beta-D-mannosylqueosine, 5-methoxycarbonylmethyluridine, 5-methoxyuridine, 2-methylthio-N6 isopentenyladenosine, N-((9-beta-D-ribofuranosyl-2-methylthiopurine-6-yl)carbamoyl)threonine, N-((9-beta-D-ribofuranosylpurine-6-yl) N-methylcarbamoyl) threonine, uridine-5-oxy acetic acid methylester uridine-5-oxyacetic acid, wybutoxosine, pseudouridine, queosine, 2-thiocytidine, 5-methyl-2-thiouridine, 2-thiouridine, 2-thiouridine, 5-methyluridine, N-((9-beta-D-ribofuranosylpurine-6-yl) carbamoyl) threonine, 2′-O-methyl-5-methyluridine, 2′-0-methyluridine, wybutosine, and 3-(3-amino-3-carboxypropyl) uridine.

The miR-34 sequences of the present invention can also be attached to a peptide or a peptidomimetic ligand which may affect pharmacokinetic distribution of the miR-34 sequence such as by enhancing cellular recognition, absorption and/or cell permeation. The peptide or peptidomimetic moiety can be about 5-50 amino acids long, e.g., about 5, 10, 15, 20, 25, 30, 35, 40, 45, or 50 amino acids long. A cell permeation peptide can also include a nuclear localization signal (NLS). For example, a cell permeation peptide can be a bipartite amphipathic peptide, such as MPG, which is derived from the fusion peptide domain of HIV-I gp41 and the NLS of SV40 large T antigen (Simeoni et al, Nucl. Acids Res. 31:2717-2724, 2003). Exemplary cell permeation peptides that may be conjugated to the miR-34 sequences of the present invention include Penetratin (RQIKIWFQNRRMKWKK, Seq. ID. 73), Tat fragment (GRKKRRQRRRPPQC, Seq. ID. 74), Signal sequence based peptide (GALFLGWLGAAGSTMGAWSQPKKKRKV, Seq. ID. 75), PVEC (LLIILRRRIRKQAH AHSK, Seq. ID. 76), Transportan (GWTLNS AGYLLKTNLKALAALAKKIL, Seq. ID. 77), Amphiphilic model peptide (KLALKLALKALKAALKLA, Seq. ID. 78), Arg9 (RRRRRRRRR, Seq. ID. 79), Cecropin PI (SWLSKTAKKLENSAKKRISEG IAIAIQGGPR, Seq. ID. 80), alpha.-defensin (ACYCRIPACIAGERRYGTCI YQGRLWAFCC, Seq. ID. 81), b-defensin (DHYNCVSSGGQCLYSACPIFTKIQGT CYRGKAKCCK, Seq. ID. 82), Bactenecin (RKCRIWIRVCR, Seq. ID. 83), PR-39 (RRRPRPPYLPR PRPPPFFPPRLPPRIPPGFPPRFPPRFPGKR-Nm, Seq. ID. 84) and Indolicidin (LPWKWPWWPWRR-NH2, Seq. ID. 85).

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MIS-containing peptide is RFGF having the amino acid sequence AAVALLPAVLLALLAP, Seq. ID. 86. An RFGF analogue (e.g., amino acid sequence AALLPVLLAAP, Seq. ID. 87) containing a hydrophobic MIS can also be a targeting moiety. The peptide moiety can be a “delivery” peptide, which can carry large polar molecules including peptides, oligonucleotides, and proteins across cell membranes. For example, sequences from the HIV Tat protein (GRKKRRQRRRPPQ, Seq. ID. 88) and the Drosophila Antennapedia protein (RQIKIWFQNRRMKWKK, Seq. ID. 89) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). The peptide or peptidomimetic which may be tethered to the miR-34 inhibitors of the invention may be a cell targeting peptide such as an arginine-glycine-aspartic acid (RGD)-peptide, or RGD mimic.

In other embodiments, the miR-34 inhibitors of the invention may be attached a cholesterol moiety, e.g., at the 3′ or 5′ end.

Accordingly, because the miR-34 containing nucleic acid molecules of the present invention may comprise or consist of either deoxyribonucleotides or ribonucleotides, it is to be understood that every miR-34 sequence that is illustrated as comprising the deoxyribonucleotides A, C, T, and G, can equally comprise the ribonucleotides A, C, U, and G, where every position that is a T in the deoxyribonucleotide is substituted with a U in the ribonucleotide version, and vice versa.

The general term miR-34 includes all members of the miR-34 family that share at least part of a mature miR-34 sequence. Mature miR-34 sequences include hsa-miR-34a UGGCAGUGUCUUAGCUGGUUGUU (MIMAT0000255; SEQ ID NO:1); hsa-miR-34b UAGGC AGUGUC AUUAGCUGAUUG (MIMAT0000685; SEQ ID NO:2); hsa-miR-34c AGGCAGUGUAGUUAGCUGAUUGC (MIMAT0000686; SEQ ID NO:3); cbr-miR-34 AGGCAGUGUGGUUAGCUGGUUG (MIMAT0000466; SEQ ID NO:4); rno-miR-34b UAGGC AGUGUAAUUAGCUGAUUG (MIMAT0000813; SEQ ID NO:5); dps-miR-34 UGGCAGUGUGGUUAGCUGGUUG (MIMATOOO1223; SEQ ID NO:6); cel-miR-34 AGGCAGUGUGGUUAGCUGGUUG (MIMAT0000005; SEQ ID NO:7); mml-miR-34a UGGCAGUGUCUUAGCUGGUUGU (MIMAT0002499; SEQ ID NO: 8); mmu-miR-34b AGGC AGUGUAAUUAGCUGAUUGU (MIMAT0000382; SEQ ID NO:9); sla-miR-34a UGGCAGUGUCUUAGCUGGUUGU (MIMAT0002500; SEQ ID NO: 10); ppy-miR-34a UGGCAGUGUCUUAGCUGGUUGU (MIMAT0002497; SEQ ID NO: 11); bta-miR-34c AGGCAGUGUAGUUAGCUGAUUG (MIMAT0003854; SEQ ID NO: 12); dre-miR-34c AGGCAGUGCAGUUAGUUGAUUAC (MIMAT0003759; SEQ ID NO: 13); mmu-miR-34a UGGCAGUGUCUUAGCUGGUUGU (MIMAT0000542; SEQ ID NO: 14); rno-miR-34a UGGCAGUGUCUUAGCUGGUUGUU (MIMAT0000815; SEQ ID NO: 15); bta-miR-34b AGGC AGUGUAAUUAGCUGAUUG (MIMAT0003549; SEQ ID NO: 16); dme-miR-34 UGGCAGUGUGGUUAGCUGGUUG (MIMAT0000350; SEQ ID NO: 17); ggo-miR-34a UGGCAGUGUCUUAGCUGGUUGU (MIMAT0002494; SEQ ID NO: 18); mdo-miR-34a UGGCAGUGUCUUAGCUGGUUGUU (MIMAT0004096; SEQ ID NO: 19); gga-miR-34a UGGCAGUGUCUUAGCUGGUUGUU (MIMATOOO1173; SEQ ID NO:20); age-miR-34a UGGCAGUGUCUUAGCUGGUUGU (MIMAT0002495; SEQ ID NO:21); gga-miR-34b CAGGCAGUGUAGUUAGCUGAUUG (MIMATOOOI 179; SEQ ID NO:22); IIa-miR-34a UGGCAGUGUCUUAGCUGGUUGU (MIMAT0002501; SEQ ID NO:23); gga-miR-34c AGGCAGUGUAGUUAGCUGAUUGC (MIMATOOOI 180; SEQ ID NO:24); xtr-miR-34b CAGGCAGUGUAGUUAGCUGAUUG (MIMAT0003579; SEQ ID NO:25); ppa-miR-34a UGGCAGUGUCUUAGCUGGUUGU (MIMAT0002496; SEQ ID NO:26); mmu-miR-34c AGGCAGUGUAGUUAGCUGAUUGC (MIMAT0000381; SEQ ID NO:27); dre-miR-34 UGGCAGUGUCUUAGCUGGUUGU (MIMATOOO1269; SEQ ID NO:28); xtr-miR-34a UGGCAGUGUCUUAGCUGGUUGUU (MIMAT0003578; SEQ ID NO:29); bmo-miR-34 UGGCAGUGUGGUUAGCUGGUUG (MIMAT0004197; SEQ ID NO:30); dre-miR-34b UAGGCAGUGUUGUUAGCUGAUUG (MIMAT0003346; SEQ ID NO:31); rno-miR-34c AGGCAGUGUAGUUAGCUGAUUGC (MIMAT0000814; SEQ ID NO:32); mne-miR-34a UGGCAGUGUCUUAGCUGGUUGU (MIMAT0002502; SEQ ID NO:33); ptr-miR-34a UGGCAGUGUCUUAGCUGGUUGU (MIMAT0002498; SEQ ID NO:34) or a complement thereof. In certain aspects, a subset of these miRNAs will be used that include some but not all of the listed miR-34 family members. The term miR-34 includes all members of the miR-34 family unless specifically identified. In certain aspects, a subset of these miRNAs will be used that include some but not all of the listed miR-34 family members.

In a further aspect, a “miR-34 nucleic acid sequence” includes all or a segment of the full length precursor of miR-34 family members. Stem-loop sequences of miR-family members include hsa-mir-34a GGCCAGCUGUGAGUGUUUCUUUGGCAGUGUCUUAGCUGGUUGUUGUGA GCAAUAGUAAGGAAGCAAUCAGCAAGUAUACUGCCCUAGAAGUGCUGC ACGUUGUGGGGCCC (MI0000268; SEQ ID NO:35); hsa-mir-34b GUGCUCGG UUUGUAGGCAGUGUCAUUAGCUGAUUGUACUGUGGUGGUUACAAUCAC UAACUCCACUGCCAUCAAAACAAGGCAC (MI0000742; SEQ ID NO:36); hsa-mir-34c AGUCUAGUUACUAGGCAGUGUAGUUAGCUGAUUGCUAAUAGUACCAAU CACUAACCACACGGCCAGGUAAAAAGAUU (MI0000743; SEQ ID NO:37); gga-mir-34c AGCCUGGUUACCAGGCAGUGUAGUUAGCUGAUUGCCACCAGGACCAA UCACUAACCACACAGCCAGGUAAAAAG (MI0001261; SEQ ID NO:38); xtr-mir-34b UUCAGGCAGUGUAGUUAGCUGAUUGUGUUAUAUCAAAUUUGCAAU CACUAGCUAAACUACCAUAAAA (MI0004818; SEQ ID NO:39); age-mir-34a GGCCAGCUGUGAGUGUUUCUUUGGCAGUGUCUUAGCUGGUUGUUGUGU GCAAUAGUGAAGGAAGCAAUCAGCAAGUAUACUGCCCUAGAAGUGCUG CACGUUGUGGGGCCC (MI0002797; SEQ ID NO:40); ptr-mir-34a GGCCAGCUGUGAGUGUUUCUUUGGCAGUGUCUUAGCUGGUUGUUGUGA GCAAUAGUAAGGAAGCAAUCAGCAAGUAUACUGCCCUAGAAGUGCUGC ACGUUGUGGGGCCC (MI0002800; SEQ ID NO:41); bta-mir-34b GUGCUCGGUUUGUAGGCAGUGUAAUUAGCUGAUUGUACUCUCAUGCUU ACAAUCACUAGUUCCACUGCCAUCAAAACAAGGCAC (MI0004763; SEQ ID NO:42); mne-mir-34a GGCCAGCUGUGAGUGUUUCUUUGG CAGUGUCUUAGCUGGUUGUUGUGAGCAAUAGUAAGGAAGCAAUCAGCA AGUAUACUGCCCUAGAAGUGCUACACAUUGUGGGGCCU (MI0002804; SEQ ID NO:43); gga-mir-34b GUGCUUGGUUUGCAGGCAGUGUAGUUAGCUG AUUGUACCCAGCGCCCCACAAUCACUAAAUUCACUGCCAUCAAAACAAG GCAC (MIOOO1260; SEQ ID NO:44); rno-mir-34c AGUCUAGUUACUAGG CAGUGUAGUUAGCUGAUUGCUAAUAGUACCAAUCACUAACCACACAGCC AGGUAAAAAGACU (MI0000876; SEQ ID NO:45); xtr-mir-34b UUCAGGCAGUGUAGUUAGCUGAUUGUGUUAUAUCAAAUUUGCAAUCACUA GCUAAACUACCAUAAAA (MI0004817; SEQ ID NO: 46); xtr-mir-34a CUGUGAGUGUU UCUUUGGCAGUGUCUUAGCUGGUUGUUGUGGCACGUUAUAGAAGUAGC AAUCAGCAAAUAUACUGCCCUAGAAGUUCUGCACAUU (MI0004816; SEQ ID NO:47); mrnu-mir-34c AGUCUAGUUACUAGGCAGUGUAGUUAGCUGAUUG CUAAUAGUACCAAUCACUAACCACACAGCCAGGUAAAAAGACU (MI0000403; SEQ ID NO:48); Ila-mir-34a GGCCAGCUGUGAGUGUUUCUUUGGCAGUGUCUU AGCUGGUUGUUGUGAGCAAUAGUGAAGGAAGCAAUCAGCAAGUAUACU GCCCUAGAAGUGCUGCACGUUGUGGGGCCC (MI0002803; SEQ ID NO:49); bmo-mir-34 AGAAUCAGGGUAGACCGCGUUGGCAGUGUGGUUAGCUGGUUGUG UAUGGAAAUGACAACAGCCACUAACGACACUGCUCCUGCGUGCACCCUA AAUCA (MI0004975; SEQ ID NO:50); sla-mir-34a GGCCGGCU GUGAGUGUUUCUUUGGCAGUGUCUUAGCUGGUUGUUGUGAGCAAUAGU GAAGGAAGCAAUCAGCAAGUAUACUGCCCUAGAAGUGCUGCACGUUGU GGGGCCC (MI0002802; SEQ ID NO:51); dre-mir-34c UGCUGUGUGGUCA CCAGGCAGUGCAGUUAGUUGAUUACAAUCCAUAAAGUAAUCACUAACC UCACUACCAGGUGAAGGCUAGUA (MI0004774; SEQ ID NO:52); rno-mir-34b GUGCUCGGUUUGUAGGCAGUGUAAUUAGCUGAUUGUAGUGCGGUGCUG ACAAUCACUAACUCCACUGCCAUCAAAACAAGGCAC (MI0000875; SEQ ID NO:53); mdo-mir-34a GGCCAGCUGUGAGUGUUUCUUUGGCAGUGUCUUAGCU GGUUGUUGUGAGUAAUAGAUAAGGAAGCAAUCAGCAAGUAUACUGCCC UAGAAGUGCUGCACGUUGUUAGGCCC (MI0005280; SEQ ID NO:54); ggo-mir-34a GGCCAGCUGUGAGUGUUUCUUUGGCAGUGUCUUAGCUGGUUGUUGUGA GCAAUAGUAAGGAAGCAAUCAGCAAGUAUACUGCCCUAGAAGUGCUGC ACGUUGUGGGGCCC (MI0002796; SEQ ID NO:55); mml-mir-34a GGCCAGC UGUGAGUGUUUCUUUGGCAGUGUCUUAGCUGGUUGUUGUGAGCAAUAG UAAGGAAGCAAUCAGCAAGUAUACUGCCCUAGAAGUGCUACACAUUGU GGGGCCU (M10002801; SEQ ID NO:56); dre-mir-34b GGGGUUGGU CUGUAGGCAGUGUUGUUAGCUGAUUGUUUCAUAUGAACUAUAAUCACU AACCAUACUGCCAACACAACAACCUACA (M10003690; SEQ ID NO:57); dre-mir-34 CUGCUGUGAGUGGUUCUCUGGCAGUGUCUUAGCUGGUUGUUGUGUGGA GUGAGAACGAAGCAAUCAGCAAGUAUACUGCCGCAGAAACUCGUCACCU U (MI0001365; SEQ ID NO:58); mmu-mir-34a CCAGCUGUGA GUAAUUCUUUGGCAGUGUCUUAGCUGGUUGUUGUGAGUAUUAGCUAAG GAAGCAAUCAGCAAGUAUACUGCCCUAGAAGUGCUGCACAUUGU (MI0000584; SEQ ID NO:59); ppa-mir-34a GGC C AGCUGUG AGUGUUUCUUUG GCAGUGUCUUAGCUGGUUGUUGUGAGCAAUAGUAAGGAAGCAAUCAGC AAGUAUACUGCCCUAGAAGUGCUGCACGUUGUGGCCCCC (MI0002798; SEQ ID NO:60); rno-mir-34a CCGGCUGUGAGUAAUUCUUUGGCAGU GUCUUAGCUGGUUGUUGUGAGUAUUAGCUAAGGAAGCAAUCAGCAAGUAU ACUGCCCUAGAAGUGCUGCACGUUGU (MI0000877; SEQ ID NO:6I); xtr-mir-34b-1 UGUUGGGUUUUCAGGCAGUGUAGUUAGCUGAUUGUGUUAACAUAAGACUU GCA AUCACUAGCUAAACUACCAGCAAAACUAAACA (MI0004925; SEQ ID NO:62); ppy-mir-34a GGCCAGCUGUGAGUGUUUCUUUGGCA GUGUCUUAGCUGGUUGUUGUGAGCAAUAGUAAGGAAGCAAUCAGCAAGUA UACUGCCCUAGAAGUGCUGCACGUUGUGGGGCCC (MI0002799; SEQ ID NO:63); xtr-mir-34b-3 UGUUGGGUUUUCAGGCAGUGUAGUUAGCUGAUUGUGUUAACAUAA GACUUGCAAUCACUAGCUAAACUACCAGCAAAACUAAACA (MI0004924; SEQ ID NO:64); cbr-mir-34 AAGCACUCAUGGUCGUGAGGCAGUGUGGUUAGCUGGUUG CAUACACAGGUUGACAACGGCUACCUUCACUGCCACCCCGAACAUGUAG UCCUC (MI0000494; SEQ ID NO:65); gga-mir-34a GCCAGCUGUGA GUGUUUCUUUGGCAGUGUCUUAGCUGGUUGUUGUGAGCAAUAGUUAAG GAAGCAAUCAGCAAGUAUACUGCCCUAGAAGUGCUACACAUUGUUGGG CC (MIOOO1251; SEQ ID NO:66); bta-mir-34c AGUCUAGUU ACUAGGCAGUGUAGUUAGCUGAUUGCUAAUAAUACCAAUCACUAACCA CACGGCCAGGUAAAAAGAUU (MI0005068; SEQ ID NO:67); dps-mir-34 AAUUGGCUAUGCGCUUUGGCAGUGUGGUUAGCUGGUUGUGUAGCCAAAAU AUU GCCUUUGACCAUUCACAGCCACUAUCUUCACUGCCGCCGCGACAAGC (MI0001317; SEQ ID NO:68); dme-mir-34 AAUUGGCUAUGCGCUUUGGC AGUGUGGUUAGCUGGUUGUGUAGCCAAUUAUUGCCGUUGACAAUUCAC AGCCACUAUCUUCACUGCCGCCGCGACAAGC (MI0000371; SEQ ID NO:69); mmu-mir-34b GUGCUCGGUUUGUAGGCAGUGUAAUUAGCUGAUUGUAGUGCGG UGCUGACAAUCACUAACUCCACUGCCAUCAAAACAAGGCAC (MI0000404; SEQ ID NO:70); cel-mir-34 CGGACAAUGCUCGAGAGGCAGUGUGGUUA GCUGGUUGCAUAUUUCCUUGACAACGGCUACCUUCACUGCCACCCCGAA CAUGUCGUCCAUCUUUGAA (MI0000005; SEQ ID NO:71) or a complement thereof.

In certain aspects, a nucleic acid miR-34 nucleic acid, or a segment or a mimetic thereof, will comprise 5, 6, 7, 8, 9, 10, 11, 12, 13, 14, 15, 16, 17, 18, 19, 20, 21, 22, 23, 24, 25, 26, 27, 28, 29 or more nucleotides of the precursor miRNA or its processed sequence, including all ranges and integers there between. In certain embodiments, the miR-34 nucleic acid sequence contains the full-length processed miRNA sequence and is referred to as the “miR-34 full-length processed nucleic acid sequence.” In still further aspects, a miR-34 comprises at least one 5, 6, 7, 8, 9, 10, 11, 12, 13, 14, 15, 16, 17, 18, 19, 20, 21, 22, 23, 24, 25, 50 nucleotide (including all ranges and integers there between) segment of miR-34 that is at least 75, 80, 85, 90, 95, 98, 99 or 100% identical to SEQ ID NOs provided herein.

In other embodiments of the invention, there is a synthetic miRNA or inhibitor in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

It is contemplated that synthetic miRNA of the invention have one or more of the replacement, sugar modification, or noncomplementarity designs. In certain cases, synthetic RNA molecules have two of them, while in others these molecules have all three designs in place.

The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides.

When the RNA molecule is a single polynucleotide, there can be a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 residues in length, or any range derivable therein. In certain embodiments, the linker is between 3 and 30 residues (inclusive) in length.

In addition to having a miRNA or inhibitor region and a complementary region, there may be flanking sequences as well at either the 5′ or 3′ end of the region. In some embodiments, there is or is at least 1, 2, 3, 4, 5, 6, 7, 8, 9, 10 nucleotides or more, or any range derivable therein, flanking one or both sides of these regions.

Methods of the invention include reducing or eliminating activity of one or more miRNAs in a cell comprising introducing into a cell a miRNA inhibitor; or supplying or enhancing the activity of one or more miRNAs in a cell. The present invention also concerns inducing certain cellular characteristics by providing to a cell a particular nucleic acid, such as a specific synthetic miRNA molecule or a synthetic miRNA inhibitor molecule. However, in methods of the invention, the miRNA molecule or miRNA inhibitor need not be synthetic. They may have a sequence that is identical to a naturally occurring miRNA or they may not have any design modifications. In certain embodiments, the miRNA molecule and/or the miRNA inhibitor are synthetic, as discussed above.

The particular nucleic acid molecule provided to the cell is understood to correspond to a particular miRNA in the cell, and thus, the miRNA in the cell is referred to as the “corresponding miRNA.” In situations in which a named miRNA molecule is introduced into a cell, the corresponding miRNA will be understood to be the induced or inhibited miRNA function. It is contemplated, however, that the miRNA molecule introduced into a cell is not a mature miRNA but is capable of becoming or functioning as a mature miRNA under the appropriate physiological conditions. In cases in which a particular corresponding miRNA is being inhibited by a miRNA inhibitor, the particular miRNA will be referred to as the “targeted miRNA.” It is contemplated that multiple corresponding miRNAs may be involved. In particular embodiments, more than one miRNA molecule is introduced into a cell. Moreover, in other embodiments, more than one miRNA inhibitor is introduced into a cell. Furthermore, a combination of miRNA molecule(s) and miRNA inhibitor(s) may be introduced into a cell. The inventors contemplate that a combination of miRNA may act at one or more points in cellular pathways of cells with aberrant phenotypes and that such combination may have increased efficacy on the target cell while not adversely effecting normal cells. Thus, a combination of miRNA may have a minimal adverse effect on a subject or patient while supplying a sufficient therapeutic effect, such as amelioration of a condition, growth inhibition of a cell, death of a targeted cell, alteration of cell phenotype or physiology, slowing of cellular growth, sensitization to a second therapy, sensitization to a particular therapy, and the like.

Methods include identifying a cell or patient in need of inducing those cellular characteristics. Also, it will be understood that an amount of a synthetic nucleic acid that is provided to a cell or organism is an “effective amount,” which refers to an amount needed (or a sufficient amount) to achieve a desired goal, such as inducing a particular cellular characteristic(s).

In certain embodiments of the methods include providing or introducing to a cell a nucleic acid molecule corresponding to a mature miRNA in the cell in an amount effective to achieve a desired physiological result.

Typically, miRNA is modulated in the cell. In particular embodiments, the nucleic acid sequence comprises at least one segment that is at least 70, 75, 80, 85, 90, 95, or 100% identical in nucleic acid sequence to one or more miRNA or gene sequence. Modulation of the expression or processing of an endogenous gene, miRNA, or mRNA can be through modulation of the processing of a mRNA, such processing including transcription, transportation and/or translation with in a cell. Modulation may also be effected by the inhibition or enhancement of miRNA activity with a cell, tissue, or organ. Such processing may affect the expression of an encoded product or the stability of the mRNA. In still other embodiments, a nucleic acid sequence can comprise a modified nucleic acid sequence. In certain aspects, one or more miRNA sequence may include or comprise a modified nucleobase or nucleic acid sequence.

In certain embodiments, methods also include targeting a miRNA to modulate in a cell or organism. The term “targeting a miRNA to modulate” means a nucleic acid of the invention will be employed so as to modulate the selected miRNA. In some embodiments the modulation is achieved with a synthetic or non-synthetic miRNA that corresponds to the targeted miRNA, which effectively provides the targeted miRNA to the cell or organism (positive modulation). In other embodiments, the modulation is achieved with a miRNA inhibitor, which effectively inhibits the targeted miRNA in the cell or organism (negative modulation).

In some embodiments, the miRNA targeted to be modulated is a miRNA that affects a disease, condition, or pathway. In certain embodiments, the miRNA is targeted because a treatment can be provided by negative modulation of the targeted miRNA. In other embodiments, the miRNA is targeted because a treatment can be provided by positive modulation of the targeted miRNA or its targets.

In another preferred embodiment, the compound is an oligopeptide or an chemically modified oligopeptide, in particular, a protein, an enzyme, an antibody or a fragment of an antibody. The skilled person is well aware of suitable compounds useful for targeting the miRNA to interfere between the miRNA and/or reduce the expression of said miRNA or antagonise the activity of said miRNA. In a particularly preferred embodiment, the compound is an antisense miRNA compound and an oligomeric compound of 50 to 49 nucleic basis in length.

In another preferred embodiment, the compound is a nucleotide sequence having at least 90% sequence complimentarity, preferably at least 95% sequence complimentarity, to a target region within the miRNA or precursor thereof, like pre-miRNA or pri-miRNA, in particular, has at least 100% sequence complimentarity to the miR-34 seed sequence. That is, the compound useful for administering to a subject for preventing or treating memory impairment and/or a neurodegenerative condition is an inhibitor which specifically inhibits the miRNA function.

The method according to the present invention is particularly useful for treating or preventing memory impairment and/or a neurodegenerative condition, disease or disorder whereby said condition, disease or disorder is a memory impairment condition selected from brain trauma, strokes, meningitis, epilepsy, subjective mild cognitive impairment, mild cognitive impairment. In addition, it is preferred that the neurodegenerative condition is a is a dementia, in particular, Alzheimer's disease HIV-related dementia, frontotemporal dementia, vascular dementia, Lewy body disease, mixed dementias, familial Danish Dementia, familial British Dementia, inclusion body myositis (IBM), or neuronal disorder related to aging processes.

In another preferred embodiment, the disease or disorder is a neuropsychiatric condition, such as anxiety disorders or depression disorders, in particular, posttraumatic stress disorder, phobia, major depressive disorder, schizophrenia. The administration of the compounds according to the present invention may be effected by the stereotactic injection, intravenously, orally, nasally, intracranially, intramuscularly, or by injection into the cerebrospinal fluid.

The compounds may be administered with a vehicle for promoting size specific delivery. Said vehicle useful for delivery of the compounds, e.g. for delivery over the blood-brain barrier, may be cholesterol-tag or other lipidtags or bio functionalized (e.g. antibody-tag) artificial particles, like polybutylcyanoacrylate (PBCA)-stabilized microbubbles, exosomes or nano-or microparticles mediating cell specific drug delivery containing said compound for delivery thereof.

Another possibility to pass the blood-brain barrier may be the use of exosomes as described for example in Alvarez-Erviti L, et al., Nat. Biotechnol. 2011 April; 29(4):341-5.

In another aspect, the present invention relates to a method for improving memory functionalability in a subject suffering from memory loss. Said method comprises reducing the level of miR-34 levels or precursor molecule levels in brain tissue of the subject by administering to said subject a therapeutically effective amount of a compound targeting miR-34 or any precursor thereof, or a miRNA sharing the same miRNA seed with miR-34 or any precursor thereof.

In addition, recognizing that miRNA or precursors thereof are relevant for memory allows for diagnosing or predicting memory impairment and/or a neurodegenerative condition, disease or disorder in a subject thereof. By determining the level of expression of miR-34 or any precursor thereof, it is possible to diagnose or predict memory impairment and/or a neurodegenerative condition as identified above. In particular, said method for diagnosing or predicting according to the present invention allows to diagnose or predict any diseases selected from brain trauma, strokes, meningitis, epilepsy, subjective mild cognitive impairment, mild cognitive impairment, Alzheimer's disease HIV-related dementia, frontotemporal dementia, vascular dementia, Lewy body disease, mixed dementias, familial Danish Dementia, familial British Dementia, inclusion body myositis (IBM), or neuronal disorder related to aging processes, neuropsychiatric condition, such as anxiety disorders or depression disorders, in particular, posttraumatic stress disorder, phobia, major depressive disorder, schizophrenia.

The method for diagnosing or predicting the memory impairment and/or neurodegenerative condition, disease or disorder comprises the steps of determining the level of expression of miR-34c in a biological sample, like in tissue, blood, liquor or cerebrospinal fluid sample
comparing the level of expression of miR-34 or any precursor thereof, in particular, miR-34c, with a reference sample of a subject not afflicted with or suspected to have memory impairment and/or a neurodegenerative condition, disease or disorder;
predicting or diagnosing memory impairment and/or a neurodegenerative condition, disease or disorder based on the expression level of miR-34 or any precursor thereof, in particular, based on miR-34c expression level in the biological sample, like in tissue, blood, liquor or cerebrospinal fluid. It is particularly preferred determining the level of the miR molecule in liquor.
   That is, it has been recognized that miR-34, an in particular, miR-34c levels can be diluent in the liquor of individuals.

Moreover, the present invention provides a method for inducing memory loss in an individual comprising administering to said individual an effective amount of one or more compounds having the functionality of the small-noncoding miR-34, in particular miR-34c or any precursor thereof or a miRNA sharing the same miRNA seed with miR-34, in particular, administering miRNA-34c or any precursor thereof.

That is, as demonstrated herein, by administering compounds having the functionality of the small non-coding miR-34 in particular, by administering miR-34c or any precursor thereof or a miRNA sharing the same miRNA seed, and, thus, interfering with the same target of miR-34 in particular miR-34c, allows to induce memory loss in an individual. The memory loss is preferably due to neuropsychiatric condition involving pathological memories like excessive fear memory as in anxiety disorders or depression disorders. In particular, said neuropsychiatric condition where the compounds having the functionality of small non-coding miR-34, in particular, miR-34c may be administered is selected from posttraumatic stress disorder, phobia, major depressive disorder, schizophrenia.

In another aspect, the present invention relates to a pharmaceutical composition containing a compound targeting miR-34, pre-miR-34 precursor, pri-miR-34 precursor, or a miRNA sharing the same miRNA seed with miR-34, in particular, targeting miR-34c or any precursor thereof together with a pharmaceutically acceptable carrier, diluent or recipient.

The active ingredient of the pharmaceutical composition to the present invention may be present in form of naked oligonucleotides or lipid membrane vessels, like liposomes or exosomes, or polybutylcyanoacrylate (PBCA)-stabilized micro- or nanobubbles or micro- or nanoparticles.

In particular, the pharmaceutically acceptable carrier are lipids, polymers, lipopolyamines, in particular, small lipid bodies, e.g. lipid nanoparticles or nanotubes, liposomes, exosomes.

The pharmaceutical composition according to the present invention is particularly useful for treating or preventing memory impairment and/or a neurodegenerative condition, disease or disorder in a subject in need thereof, in particular, to prevent or treat any of the specifically identified diseases, disorders, or conditions described herein.

In addition, the present invention relates to the use of miRNA for memory loss in an individual, in particular, in an individual suffering from a neuropsychiatric condition involving pathological memories, like excessive fear memory as in anxiety disorders or depression disorders. Particularly useful compounds for inducing memory loss include miR-34c or any precursor thereof or a miRNA sharing the same miRNA seed with miR-34, in particular, miR-34c.

### EXAMPLES

### Materials and Methods

### Animals

Mice (C57Bl/6J) were housed with free access to food and water under standard light/dark conditions (12 h-12 h). As a model for Alzheimer's disease the present inventors employed the double transgenic APPPS1-21 mouse co-expressing the KM670/671NL “Swedish” mutation of the amyloid precursor protein (APP) and the L166P mutation of the presenility 1 (PSI) gene, on C57BL/6J strain background. This mouse exhibits an aggressive β-amyloid pathology and cognitive deficits at an early age. All experiments were carried out in accordance with the animal protection law and were approved by the District Government of Germany.

### Cannulae Implants and Hippocampal Injections

Hippocampal injections were performed as described previously (Sananbenesi et al, 2007; Nat Neurosci 10(8): 1012-1019). In brief, mice were anesthetized and microcannulae were stereotactically implanted to the hippocampus (1.0 mm posterior to the bregma; 1.0 mm lateral from midline; and 1.5 mm ventral). After recovery from surgery, mice received bilateral injections (1 μl, at a rate of 0.30 min⁻¹ per side) of miRNA mimics and inhibitors at the indicated time points before FC (as indicated by arrows in the experimental schedules). Hippocampal injections used a 1.5 mm-gauge needle that extended 1.5 mm beyond the tip of the guide cannula.

### Real-Time Quantitative RT-PCR and Western Blot Analysis

Total RNA (including miRNAs) and protein was isolated from mice hippocampi (both hemispheres for each preparation) using the TRI-Reagent (Sigma-Aldrich, Steinheim, Germany) according to manufacturer's recommendations, but with the step of RNA precipitation in isopropanol extended to a total of 60 minutes and protein dissolved in 9M Urea dissolved in 1% SDS. With the exception of experiments involving injection of mimics and inhibitors (where the CA regions were specifically dissected under a stereomicroscope (Motic) as described previously (Hagihara et al, 2009; J Vis Exp 17(33): pii 1543), whole hippocampal lysates were used. Conversion of 1 μg RNA into cDNA and Real-time PCR detection of miRNAs were carried out using the miScript Reverse Transcription Kit and the miScript SYBR-Green PCR Kit (Qiagen), respectively, on a LC480 LightCycler (Roche Applied Science, Mannheim, Germany). Exact primer sequences and conditions are listed in supplemetary material. Proteins were resolved by SDS-PAGE and western blot analysis was performed as described previously (Kuczera et al, 2010), using the following antibodies: Rabbit Sirt-1 (dil. 1-10000, Millipore), mouse b-actin (dil. 1:2500, sc-69870, Santa Cruz, Calif., USA).

### Small RNA Massive Parallel Sequencing

Sequencing was performed in an Illumina GAII Genome Analyzer at Fastens SA (Plan-les-Ouates, Switzerland) using the Chrysalis 36 cycles v4.0 sequencing kit and RTA SCS v2.6 and GERALD 1.5.1. data analysis pipeline. The respective protocols are provided by Illumina (Illumina, San Diego, Calif.) at http://www.illumina.com/systems/genome_analyzer_iix.ilmn. Each of the four biological replicates representing a small RNA library from one mouse hippocampus was indexed separately. In total 24.811.899 reads representing 1.339.842.546 bases were derived, with 97.8% of the reads being attributed to a sample in average. The sequences and insert sequence files, provided in solexa fast-q format, are available through the Galaxy data sharing platform at: (http://main.g2.bx.psu.edu/u/fischerlab/h/sm—zovoilis-et-al; published history SM—).

More than 89% of sequenced reads were found within a length range of 18 to 26 nt, range covering all known miRNAs, and thus only reads within this range have been further used for mapping.

### Bioinformatics and Statistical Analysis

For computational prediction of miRNA targets, the TargetScan web platform (www.targetscan.com/release 5.1) has been used. For functional annotation of miRNA targets, the data mining environment provided by the DAVID platform (Huang et al, 2009; Nature Protoc 4(1): 44-57) has been used. The functional annotation module was applied for gene ontology terms and terms in PANTHER database using an EASE score of 0.1 and a minimum number of two counts. For testing enrichment in specific KEGG pathways of miRNA targets DIANA-mirPath (Papadopoulos et al, 2.009 GL. Bioinformatics doi: 10.1093/bioinformatics/btp299) has been used. The software performs an enrichment analysis of multiple microRNA target genes comparing each set of microRNA targets to all known KEGG pathways. The combinatorial effect of co-expressed microRNAs in the modulation of a given pathway is taken into account by the simultaneous analysis of multiple microRNAs (Papadopoulos et al, 2009, see above). For the analysis of the gene expression data regarding microRNA function and determination of their respective enrichment in miRNA targets DIANA-mirExTra (default running parameters) has been used, an algorithm that can identify microRNA effects to the expression levels of protein-coding transcripts, based on the frequency of six nucleotide long motifs (hexamers) in the 3′UTR sequences of genes (Alexiou et al, 2010; PLoS One 5(2): e9171). Data of qPCR, western blotting and behavioral studies are expressed as mean±SE (standard error), and unless stated otherwise significance was defined performing student's T-test.

### Contextual Fear Conditioning and Other Behavioral Tests

To assess associative learning, the contextual fear conditioning paradigm was used. Behavior testing was performed as described previously Peleg et al, 2010; Science 328 (753): 753-756 employing TSE Systems apparatuses and software and Scan software (Clever Systems). Mice were single housed and habituated to the testing room at least 3 days before behaviour experiments. The training consisted of a single exposure to the conditioning context (3 min) followed by a single electric foot shock (0.7 mA, constant current, 2s) (Fear Conditioning-FC) and the memory test was performed 24 h later (Test).

The water maze training was performed as described previously Takada S., et al. RNA 15:1507-14 (2009). In brief, in a circular tank (diameter 1.2 m) filled with opaque water a platform (11×11 cm) was submerged below the water's surface in the center of the target quadrant. Tracing the swimming pathway of the mice was performed using a video camera and the Videomot 2 software (TSE). Each training session (n=7) included the following steps:

a) mice were placed into the maze subsequently from four random points of the tank and were allowed to search for the platform for 60 s. b) In case finding of the platform was unsuccessful mice were gently guided to it. c) Mice remained on the platform for 15 s. During the memory test (probe test) mice were left to swim in the maze for 60 s with the platform removed. Time spent in the target quadrant (%) was employed to control for memory deficits in mice treated with the miRNA mimic compared with those injected with the scramble oligo.

For the novel object recognition test animals were placed in a plastic arena (100×100 cm, height 20 cm) for 5 min. The explorative behavior of the animals was recorded by a video camera and analyzed by the Videomot 2 software (TSE). During each training session (10 min), one every 24 h (n=5), mice were allowed to explore the field which in the first two sessions did not contain any object (habituation phase), the next two sessions contained two same objects (A-A), and in the last session (memory test/probe test) contained object A and a novel object B. Time spent on the novel object on that day (%) compared to the other was used to control for memory deficits in mice treated with the miRNA mimic.

### Hippocampal Injections

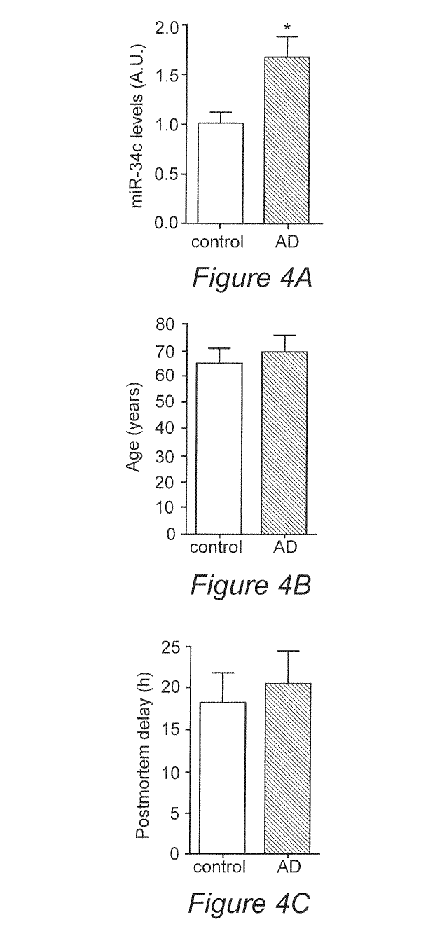

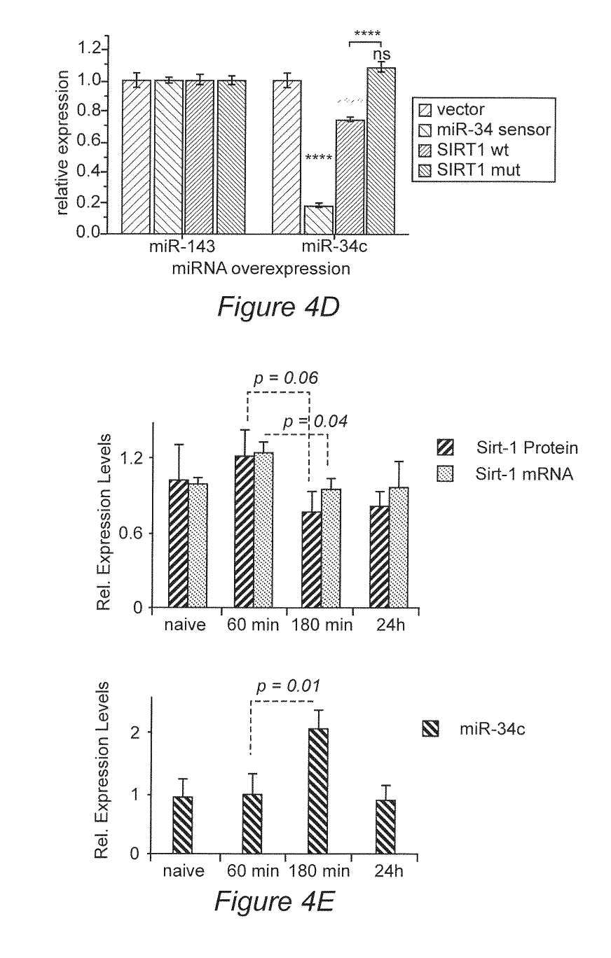

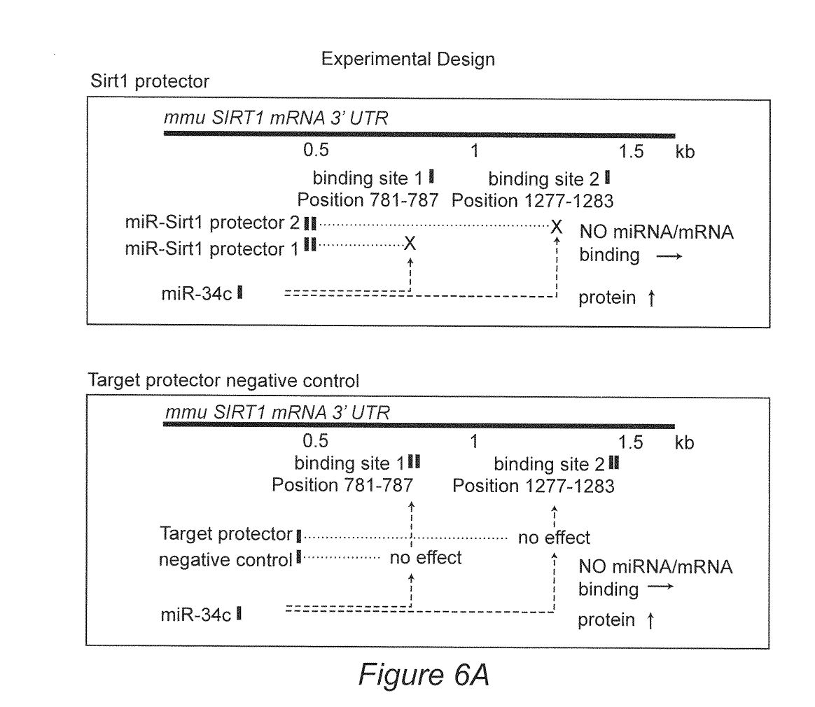

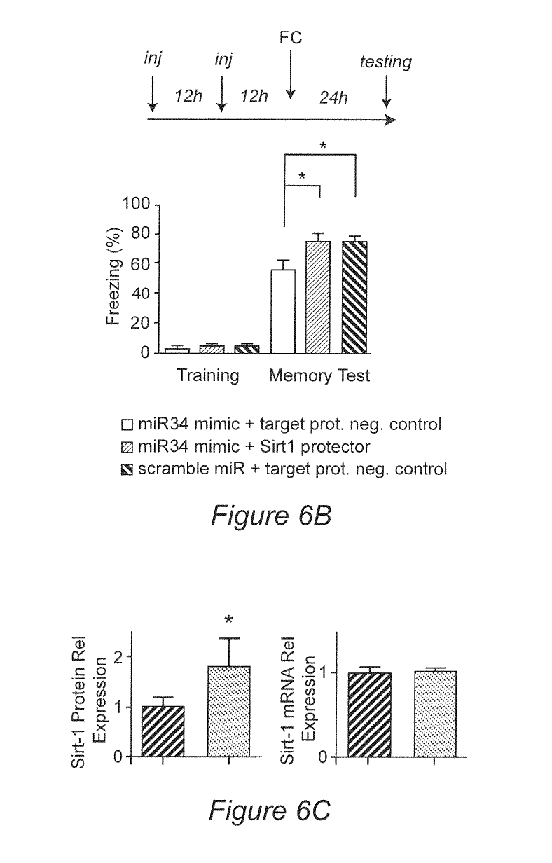

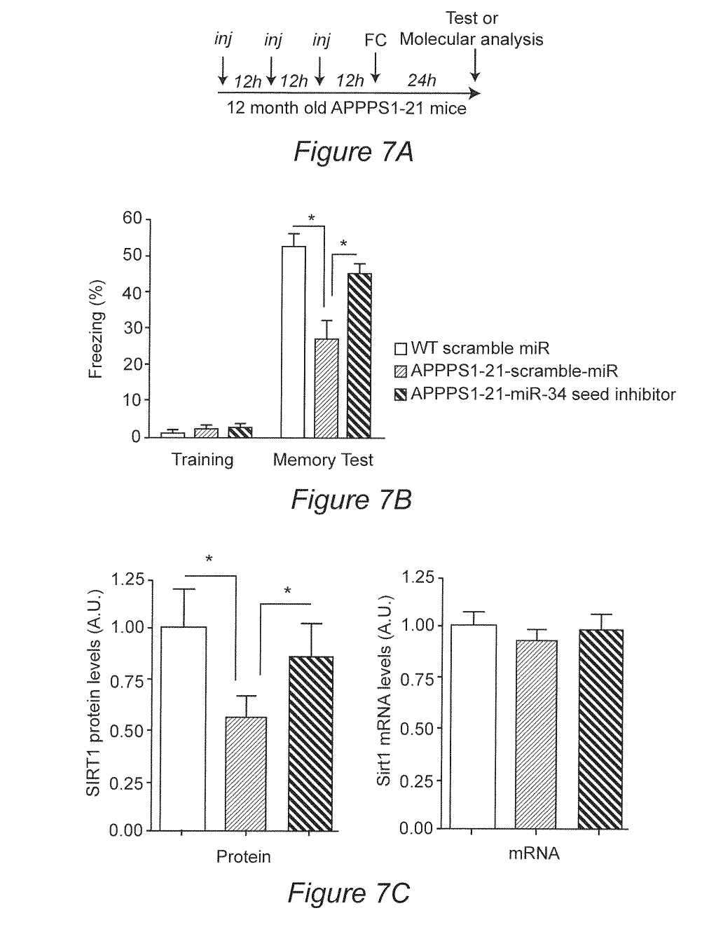

The present inventors employed miRNA mimics (synthetic RNAs which mimic mature endogenous miRNAs after transfection) for mmu-miR-34c, mature miRNA sequence: 5′ AGGCAGUGUAGUUAGCUGAUUGC (MIMAT0000384, Seq. ID No. 27) (Qiagen, Hilden, Germany). For inhibition the present inventors employed the following inhibitors (chemically synthesized modified antisense RNAs which specifically inhibit endogenous miRNA function after transfection into cells) (Qiagen) Anti-mmu-miR-34a (MIN0000542) for mmu-miR-34a, mature miRNA sequence: 5′ UGGCAGUGUCUUAGCUGGUUGU (Seq. ID No. 14), Anti-mmu-miR-346-5p (MIN0000382) for mmu-miR-34b-5p mature miRNA sequence 5r AGGCAGUGUAAUUAGCUGAUUGU (Seq. ID No. 8.), Anti-mmu-miR-34c (MIN0000381) for mmu-miR-34c mature miRNA sequence (Seq. ID No. 27). All used miRNA mimics and inhibitors were HPLC purified, for in vivo applications. For injections, miRNA mimics and inhibitors were diluted in PBS and the HiPerfect transfection reagent from Qiagen according to a modified protocol of that recommended by the manufacturer for cell transfections. In detail: Steril cold PBS (water can be also used) was added to the oligo tube to a final concentration of 100 μM for the mimic or 1 mM for the inhibitor and mixed intensively and left at room temperature for 3 minutes. All handling was done under aseptic conditions. Mixture was transferred into to 0.2 ml tubes and 1.35 μl of the HP transfection reagent was added for each 10 μl of the mimic or inhibitor mix. The rest was aliquoted and kept in −80 for not longer than a month and was not frozen-defrozen more than once. Oligo-HP mix was mixed intensively for 1 minute and left at room temperature for 5 min. Then it was warmed in the hand for 1 minute before injection. To confirm transfection of cells and successful action of oligos within the present transfection protocol in vivo: i) different mice were injected as described above with synthesized control siRNA oligo from Qiagen (Mm/Hs_MAPK1 control siRNA-Cat. No. 1027321) against Mapk1 and compared with mice injected with a validated negative control siRNA (scramble) (AllStars Neg. Control siRNA, Cat No 1027281, Qiagen). This scramble oligo has no homology to any known mammalian gene, it has been validated using Affymetrix GeneChip arrays and a variety of cell-based assays and shown to ensure minimal nonspecific effects on gene expression and phenotype. In addition, cloning experiments confirmed that it enters RISC {https://www.qiagen.com/products/genesilencing/allstarmaicontrols/allstarsnegativeco ntrols.aspx}. Reduction of mRNA levels of the target Mapk1 mRNA in the hippocampi of mice treated with the MAPK I control siRNA compared with those treated with the Neg. Control siRNA (scramble) confirmed that with the currently used transfection protocol, transfected oligos are able to enter the cell and get successfully incorporated within RISC. ii) Expression levels of the already experimentally established miR-34c target, SIRT1 were determined in mice hippocampi in comparison to mice treated with the scramble oligo and confirmed increased or reduced activity of miR-34c mimics and inhibitors, respectively (FIG. 4, FIG. 6, FIG. 7). iii) In case of transfection with miR-34c mimics expression levels of the mature miR-34c oligo were determined, confirming the increase of the total amount of the active miR-34c oligo/miRNA in mouse hippocampi of injected mice 12 h after the second injection, and immediately before FC (FIG. 4a). Importantly, mock-transfected injected mice did not present any sign of increased toxicity of the transfection agent used.

For the miRNA target protection assays the present inventors used the miScript Target Protectors, which are single-stranded, modified RNAs that specifically interfere with the interaction of an miRNA with a single target, while leaving the regulation of other targets of the same miRNA unaffected (Qiagen). The design of the protectors of SIRT1 was as follows: i) miScript Target Protector mmu_Sirt1_{—}34c_{—}780 nt for the binding site starting at position 780 nt of the Sirt1 mRNA 3′UTR: Design for: AGATCTTCACCACAAATACTGCCAAGATGTGAATATGCAA (Seq. ID No. 90). This target protector will detect several transcripts of the same gene (Sirt1):NM_{—}001159590, NM_{—}001159589, NM_{—}019812 (MTP0001486). ii) miScript Target Protector mmu_Sirt1_{—}34c_{—}1270 nt for the binding site starting at position 1270 nt of the Sirt1 mRNA 3′UTR: Design for: ACCCAGTTAGGACCATTACTGCCAGAGGAGAAAAGTATTA (Seq. ID No. 91). This target protector will detect serveral transcripts of the same gene (Sirt1):NM_{—}001159590, NM_{—}001159589, NM_{—}019812 (MTP0001493). As control the Negative Control miScript Target Protector was used (MTP0000002-Qiagen), which has no homology to any known mammalian gene. Target protectors were used according to the above described transfection and injection scheme for mimics in a final concentration of 100 μM.

### Real-Time Quantitative RT-PCR and Western Blot Analysis

The optimized miRNA-specific primers used for each miRNA as well as for the endogenous control RNU6B (miScript Primer Assays, Qiagen) are as follows: Mm_miR-34c_{—}1 (MS00001442), Mm_miR-181b_{—}1 (MS00006083), Mm_miR-379_{—}2 (MS00011942), (MS00005929), Mm_miR-30a_{—}1 (MS00011704), Hs_RNU6B_{—}2 (MS00014000). PCR specificity was checked as described previously see Zovoilis, A., et al. Mol Hum Reprod 14, 521-529 (2008). For Real-time quantitative RT-PCR of mRNAs, the LightCycler PCR Mix (Roche Applied Science) was used according to manufacturer's conditions with gene-specific primers and the respective UPL probes as follows: Sirt-1F: 5′ CGTGGAGACATTTTTAATCAGGTA (Seq. ID No. 92), Sirt-1R: 5′ GCTTCATGATGGCAAGTGG (Seq. ID No. 93), mouse UPL probe #104; Hprt-1F: 5′ TCCTCCTCAGACCGCTTTT (Seq. ID No. 94), HPRT-1 R: 5′ CCTGGTTCATCATCGCTAATC (Seq. ID No. 95) mouse UPL probe #95. Forty five cycles of PCR amplification were performed as follows: denaturation at 95° C. for 10 s, annealing at 60° C. for 30 s and extension at 72° C. for 10 s. PCR assays were performed twice for each cDNA sample. Data were analysed with the LightCycler 480 software (Roche Applied Science). The relative quantification method was chosen to determine quantities based on a standard curve of serial dilutions of a control hippocampal cDNA. All quantities were further normalized to values of RNU6B and Hprt-1 for miRNA or mRNA quantification, respectively.

For western blotting, in brief, 20 μg protein was loaded per lane of an SDS gel (consisting of a stacking gel 5% acrylamide and a running gel with 12% acrylamide concentration), it was run between 60-100V and then transferred in a wet-blot system (Mini Trans-Blot Cell, Bio-RAD) at 45-60V, 4° C. overnight onto a nitrocellulose membrane (Protran, 0.2 um, Whatman, Dassel, Germany). After transfer it was blocked in 5% milkpowder, while primary antibodies were incubated 0.5% milkpowder at 4° C. overnight, followed by three washes and incubation of with the respective fluorophore coupled secondary antibody (Li-COR Bioscienbces). Band intensity was determined in ratio to -actin.

Massive parallel sequencing and mapping of small RNAs, annotation and quantification of miRNAs.

The 3′ adapter sequences were trimmed from the raw reads in 3 steps:

(Seq. ID No. 96)
(3′ adapter sequence “ATCTCGTATGCCGTCTTCTGCTTGT”)

1) The full adapter sequence was used, which permits to identify “inserts” of 25 nt or less.

2) If no adapter sequence was found, in successive steps the last base of the adapter was removed and the sequence was searched at the end of the reads. The minimum adapter size of 8 bases permits identifying inserts of up to 42 bases.

3) Finally the remaining reads were searched for not-exact matches of the adapter. The first 5 bases of the adapter were searched within the full reads sequences and at least 80% of the following bases must be identical to the adapter sequence (max 44 bp). After trimming of the adapter sequences, the inserts were sorted in separate files according to their lengths.

For estimating the proportion of sequenced small RNAs representing miRNAs, reads were firstly mapped against the Mus Musculus Full Genome (NCBI Assembly M37) using ELAND v2 (CASAVA Version 1.6.0 from Illumina) allowing up to 2 mismatches. From the respective output files, subsequent BED files were derived and the genomic intervals defined by each read were evaluated based on whether they map on a known miRNA genomic location or not (UCSC table browser miRNA track that shows microRNAs from the miRBase (www.mirbase.org). In the latter case, the read was subtracted from the tested pool of reads. The total of the subtracted reads relative to the total number of mapped reads was used to determine the miRNA class proportion of sequenced reads and the number of the known miRNA precursor regions that are represented by at least one sequenced read in mouse hippocampus. For quantifying the reads that correspond to known mature miRNAs the present inventors used a more conservative and strict approach. Firstly, since multiple genomic locations may produce the same mature miRNA, reads were mapped not against the mouse genome but against all known mouse (mmu) mature miRNA sequences present in miRBase, which instead not show this type of redundancy. During mapping the present inventors allowed no mismatch and repressed multiple mapping reads (which due to the non-redundancy of the selected database, where almost absent). The exact Bowtie parameters were set as follows: -a , -v (allowed mismatches) to 0, -m to 1 (repressed multiple mapping reads) with the rest parameters used at default values (Bowtie 0.12.7). Then, occurrences of each mature miRNA per replicate were summed and pooled resulting in the total sequence count number, that has been further used to determine the number and the approximate range of each miRNA in mouse hippocampus. Reads derived from whole mouse brain were retrieved from see Chiang, H. R., et al. Genes Dev. 24, 992-1009 (2010) but were mapped exactly as reads from hippocampus to define the respective miRNA range in this tissue. The present inventors defined for the top ranking miRNAs in hippocampus the relative to the brain read count number as follows: the present inventors first scaled for each miRNA the count numbers in hippocampus according to the total difference in read counts between the two libraries and then normalized it to the respective count number in brain. The output was then converted to the natural logarithm of this value. In all cases the mature sequences in miRbase for each miRNA were used as the diagnostic sequence for both libraries.

miRNA Expression Constructs and Luciferase Assay

For plasmid-based expression of miR-34c the precursor hairpin was PCR-amplified from mouse genomic DNA and cloned in the 3′UTR of mCherry driven by human ubiquitin C promoter in FUW plasmid (using 5′-gatgctagcacagcttggctgacgatttgct-3′ (Seq. ID No. 97) and 5′-ataggcgcgccctatggctctgtcetcacca-3′) (Seq. ID No. 98) as described previously see Edbauer, D., et al. Neuron. 65): 373-84 (2010). For dual luciferase assays (Promega) the present inventors modified psiCHECK2 vector (Promega) to express both renilla and firefly luciferase from human synapsin promoter for robust expression in neurons. The present inventors inserted either two artificial miR-34 targets sites 5′-cgcgccAGGCAGTGTAGTTAGCTGATTGgatcc AGGCAGTGTAGTTAGCTGATTG-′3 (Seq. ID No. 99) or the mouse SIRT1 3′UTR (using 5′-ataggcgcgccactattgaagctgtccggattcagg-3′ (Seq. ID No. 100) and 5′-atagaattccagtcattaaacggtctacaaaacatatgcc-3′) (Seq. ID No. 101) in the 3′UTR of renilla luciferase. To generate a miR-34-resistant SIRT1 mutant, the present inventors removed both putative target sites for miR-34 (5′-atatcaccacaaatactgccaa-3′ (Seq. ID No. 102) and 5′-agttaggaccattactgcca-3′ (Seq. ID No. 103) using PCR-mutagenesis. For dual luciferase assays, cortical neurons were cultured from embryonic day 19 rat embryos and transfected with luciferase-based miRNA-sensors together with miRNA overexpression constructs using Lipofectamine 2000 (Invitrogen) as described previously see Edbauer, D., et al. Neuron. 65):373-84 (2010).

### Results

### Massive Parallel Sequencing Reveals the Hippocampal MicroRNAome at an Unprecedented Depth

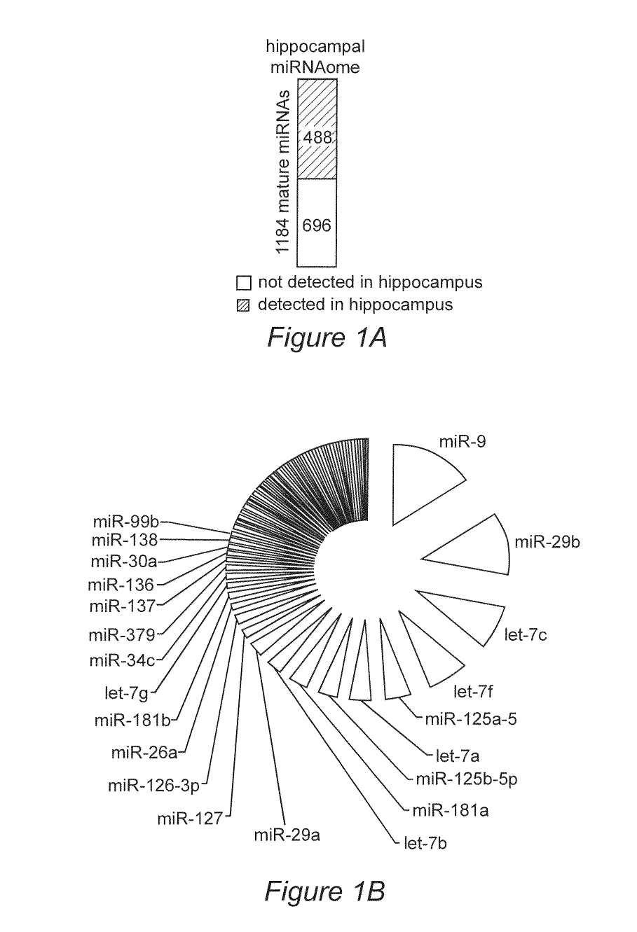

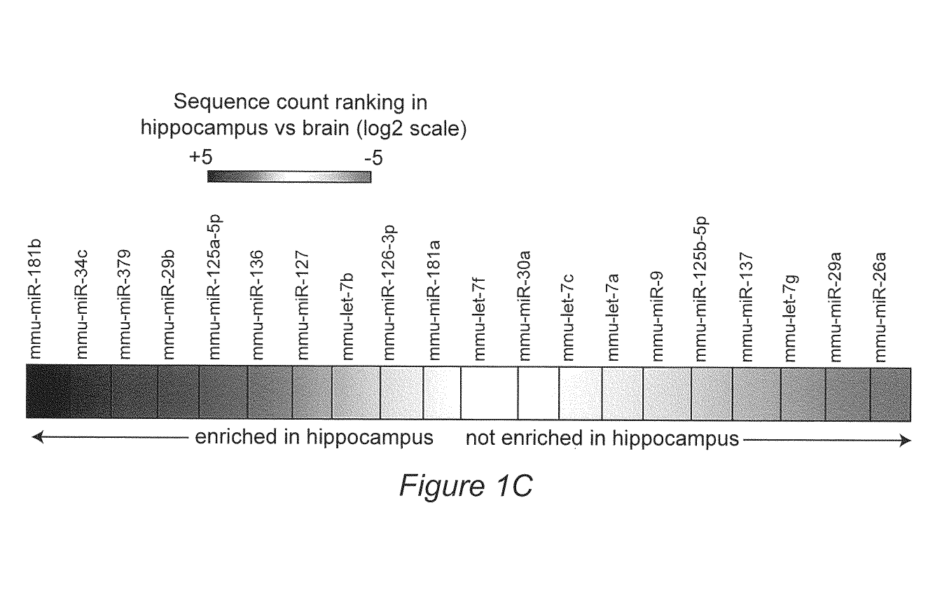

Deep sequencing of mouse hippocampal small RNA libraries derived more than 24 million reads. Previous sequencing approaches of this tissue did not exceed 100,000 sequenced clones or did not include testing and quantification of a big number of known miRNAs that were identified and characterized after these studies. From the total 1184 known mature miRNAs encoded in the mouse genome, 488 were detected in the hippocampus (FIG. 1a). Notably, out of the 488 hippocampal miRNAs, 23 miRNAs were highly expressed (beyond 100,000 counts). In fact these miRNAs accounted for almost 83% of all miRNA counts detected in hippocampal tissue (FIG. 1b).

miR-34c is Implicated with Hippocampal Function

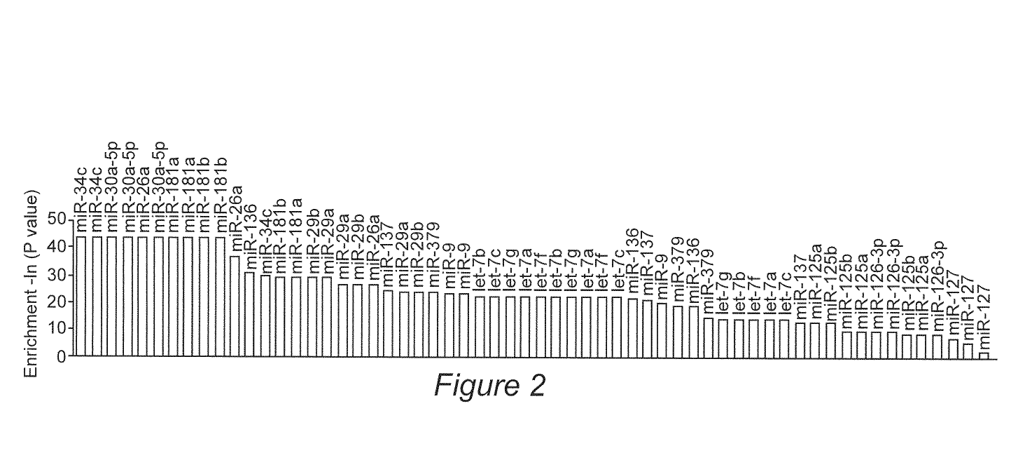

Different tissues have been shown to possess unique miRNA expression profiles in accordance to their specific structural and functional characteristics. As such it has been reasoned that a comparison of the data shown herein (FIG. 1b) with the miRNA profile of the whole mouse brain, for which comparable massive parallel sequencing data already exists (Chiang et al, 2010; Genes Dev 24(10): 992-1009), would allow to identify miRNAs that might have a unique role in hippocampal function and thus would be most suitable candidates for further analysis. The hippocampus (FIG. 1b) and whole brain tissue display many similarities regarding the most highly expressed miRNAs, like miR-9 and members of the miR-29 and let-7 families, which are implicated with the regulation of general cellular processes. However, a number of miRNAs that were minimally expressed in whole brain tissue, such as miR-181b, miR-34c or miR-379, were in contrast highly enriched in the hippocampus (FIG. 1c). Among these three miRNAs, targets of miR-34c are highly predicted to be involved in neuronal processes and functions (FIG. 2a) compared to other miRNAs tested, namely miR-181b, miR-379, miR-26a or miR-30a, suggesting involvement of miR-34c in regulation of hippocampal functions. In addition, miR-34c is transcribed from the same cluster as miR-34b.

To further test the possibility that miR-34c may be important for hippocampal function the present inventors applied an integrated genomic analysis has been applied. In a recent work it was identified 1362 hippocampal genes that are up-regulated in mice 60 min after exposure to contextual fear conditioning were identified, a commonly used paradigm for associative learning in rodents (Peleg et al, 2010 see above) It has been questioned which of the highly expressed hippocampal miRNAs identified by the deep sequencing analysis show a significant enrichment in targets within these 1362 learning associated genes. Notably, miR-34c showed the highest grade of enrichment regarding predicted targets within this group of genes (FIG. 2) suggesting a potential role for miR-34c in the regulation of memory function.

High miR-34c Levels are Linked to Pathogenesis of Learning Impairment

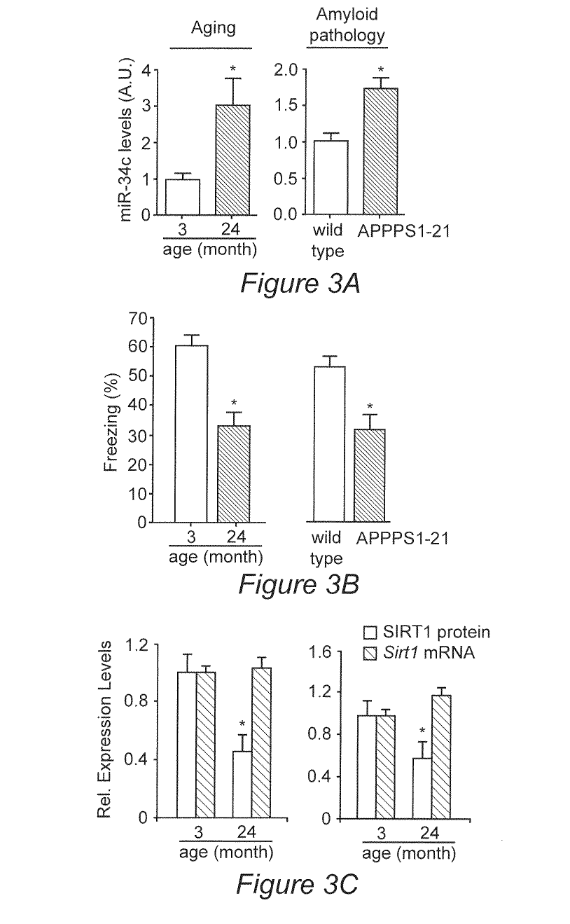

Next it has been tested whether miR-34c may play a role in memory impairment. As a first approach miR-34c levels have been measured in the hippocampus of 24-month old mice, a model for age-associated memory impairment, and in APPPS1-21 mice, a model of amyloid pathology linked to AD. The present inventors found that hippocampal miR-34c was significantly up-regulated in these two mouse models (FIG. 3a), which correlated with impaired memory function (FIG. 3b). Interestingly, among the already experimentally confirmed targets of the miR-34 family is the Sirt1 mRNA (Yamakuchi & Lowenstein, 2009; Cell Cycle 8(5): 712-715), that has two binding sites of miR-34c. As shown by the respective luciferase assays in FIG. 4d, targeting of Sirt1 mRNA by miR-34c applies also to neuronal cells. Since, hippocampal SIRT1 has been shown to be pivotal and necessary for memory function in mice (Gao et al, 2010; Nature 466(7310): 1105-1109), it has been reasoned that measuring SIRT1 levels would be a suitable read out of miR-34c activity in vivo. Indeed, learning-induced changes in SIRT1 protein levels showed an inverted correlation with the regulation of miR-34c 3 h after fear conditioning (FIG. 4e). Moreover, elevated hippocampal miR-34c levels in 24-month old mice and in APPPS1-21 mice, correlated with decreased SIRT1 protein levels (FIG. 3c). Notably, these findings in animal models translated into the human situation. To this end it has been found that miR-34c levels were significantly increased in the hippocampus of human AD patients when compared to age-matched control (FIG. 4). Within the same patients down-regulation of miR-124 has been determined that has been implicated with memory formation while the levels of miR-379 were unaffected.

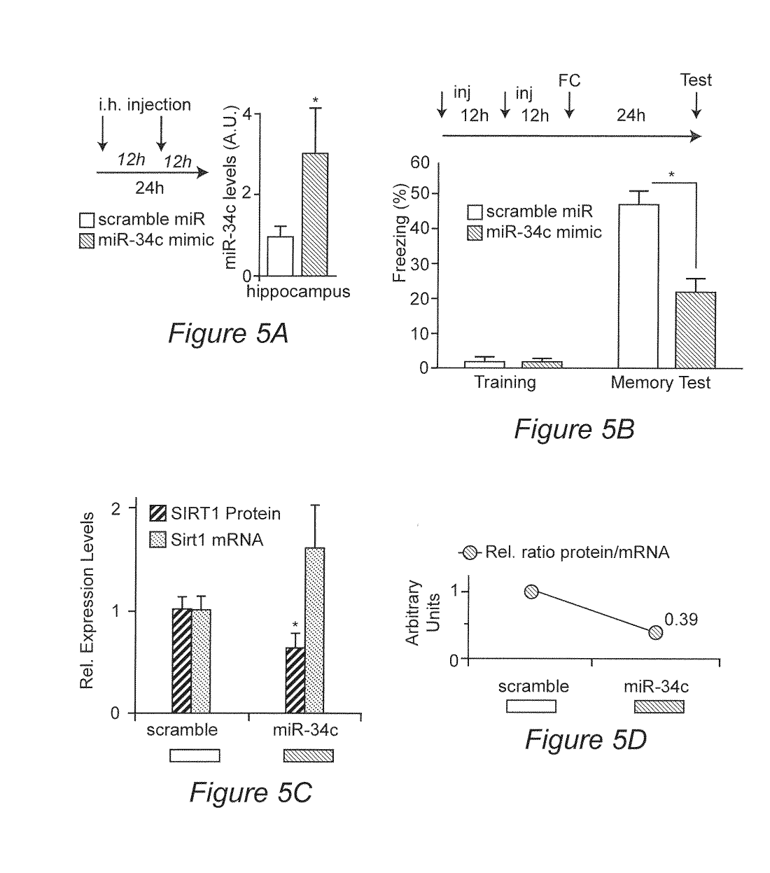

Thus, it was decided to test directly, whether elevated hippocampal miR-34c levels would affect memory function. To this end, an in vivo small RNA transfection system has been established herein (FIG. 5a) in which microinjections of a miR-34c mimic into the hippocampus resulted in significantly elevated miR-34c levels when measured 12 h after the last injection (FIG. 5a). Next, mice were injected with miR-34c mimic and subjected to fear conditioning training when hippocampal miR-34c levels were significantly elevated, thus simulating a condition similar to that observed in 24-month-old mice, APPPS1-21 mice and in human AD patients (FIG. 5a). Associative learning was significantly impaired in miR-34c mimic injected mice when compared to the scrambled miR-injected control group (FIG. 5b). miR-34c induced impairment of associative learning corresponded with reduced SIRT1 levels (FIG. 5c-d), confirming successful incorporation of miR-34c mimic into the RISC complex and specific translational repression of target mRNAs. Furthermore, specific blocking of the interaction between miR-34c and its SIRT1 3′UTR binding sites through miRNA target protectors (FIG. 6a) was able to reverse the above effects. It resulted in increased SIRT1 protein levels (FIG. 6b) and reinstated learning (FIG. 6c), confirming that action of miR-34c on SIRT1 protein in vivo is direct and based on miR-34c/mRNA interaction.

Impairment of learning upon injection of mir-34c mimic was also confirmed in two other behavioural tests, testing long term memory through novel object recognition and spatial memory formation through the Morris water maze, further supporting a role of this miRNA as a negative constraint of memory formation.

Targeting miR-34c Seed Rescues Learning Impairment

The experimental data disclosed herein strongly suggests that the miR-34c up-regulation seen in AD brains might contribute to clinical memory deficits. To test the therapeutic potential of targeting mir-34c signalling it has been considered asked whether inhibition of miR-34c seed would affect memory function in a mouse model for neurodegeneration. Different miRNAs can share the same miRNA seed. In case of miR-34c the corresponding seed is found in 6 additional miRs (miR-34a, miR34b, miR-449a/b/c and miR-699). However, the sequencing analysis shows that said miRNAs are absent or present at very low levels in the hippocampus so that miR-34c accounts for more than 98% of all reads obtained for those 6 miRNAs. To exclude any compensatory action of the remaining 2% of miR-34a and miR-34b, the inhibitors used in the present experiments targeted also these miRNAs (miR-34 seed inhibitors).

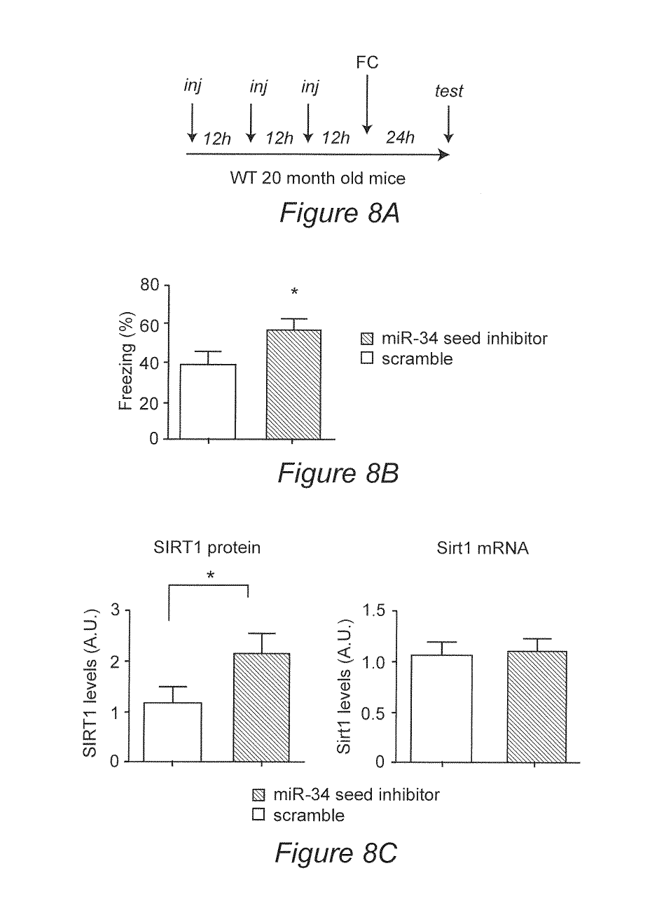

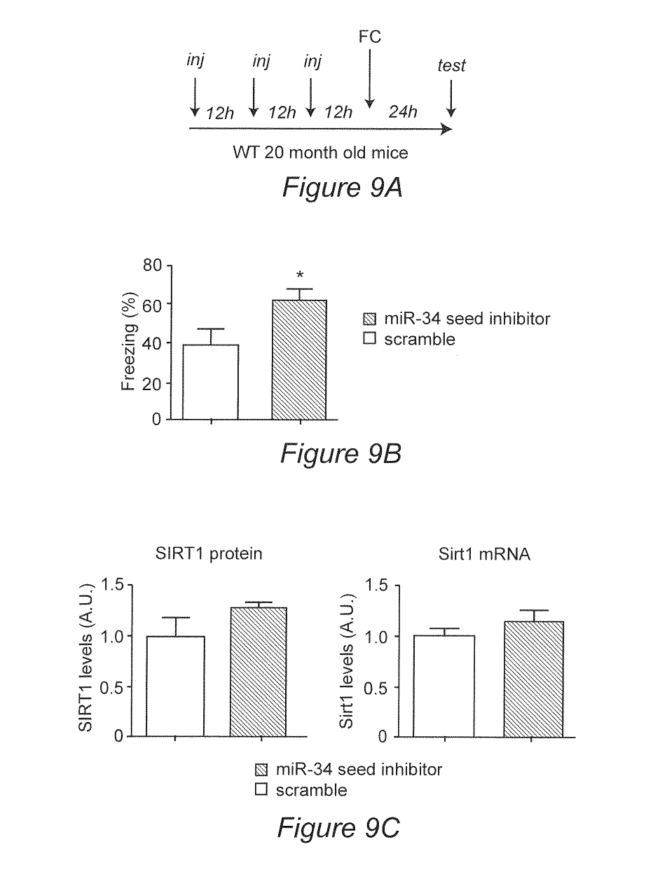

12-month-old APPPS1-21 mice has been injected with the miR-34 inhibitors (FIG. 7a). Notably, while APPPS1-21 mice injected with the scrambled oligo showed as expected impaired associative memory, miR-34 seed inhibitor treated mice exhibited a memory performance that was similar to age-matched wild-type mice (FIG. 7b). Rescued memory function correlated with restored levels of SIRT1 protein confirming functionality of miR-34 seed inhibitor in vivo (FIG. 7c). Similar effects were observed when miR-34 seed inhibitors were injected in aged mice (FIG. 8). Moreover, inhibition of miR-34 seed was able to enhance memory also in 3-month old wild type animals (FIG. 9) suggesting a general role of miR-34c as a constraint of memory formation which however might be independent from SIRT1.

### Discussion

The present invention provides a number of novel observations. First, the present inventors provide a complete and absolute digitized quantification of the mouse hippocampal miRNAome at an unprecedented depth that will be a suitable tool for further analysis is provided. Of note is the fact that a very small number of miRNAs (23 miRNAs) accounts for almost the total of miRNA reads detected in hippocampus (more than 80%). This makes the detection of low-abundance sequences difficult when only a small number of reads is produced. In addition, it makes quantification of changes in highly represented sequences equally challenging. These differences between low and high abundance miRNAs note the importance of using a high throughput approach like deep sequencing, with an unlimited fully-quantitative dynamic range of signal, in order to determine and compare hippocampal microRNAs expression profile. Thus, the current study consists the most complete and detailed representation of mouse hippocampus microRNAome until now.

Second, on the basis of the sequencing approach and integrative genomics described herein miR-34c as a microRNA linked to hippocampal function has been identified. miR-34c showed the highest grade of enrichment regarding predicted target genes within a group of hippocampal genes that are regulated during memory formation. Importantly, the present data strongly suggests that miR-34c is implicated with memory impairment in AD. It is demonstrated herein that miR-34c is up-regulated in the aging mouse hippocampus, in the hippocampus of APPPS1-21 mice and in the hippocampus of human AD patients. While the miRNA pathway involving miR-34 family has been previously studied in other biological contexts like cancer its role in the adult brain has not been addressed previously. The present data show that elevation of hippocampal miR-34c levels impairs memory function in mice, suggesting that increased miR-34c expression observed in aged mice, APPPS1-21 mice and in human AD patients contributes to memory impairment. This view is further supported by the fact that targeting miR-34c in cognitively impaired APPPS1-21 and aged wild type mice was able to improve associative learning. Of note, memory function was improved by only 3 consecutive injections of miR-34 seed inhibitors within 36 h, indicating that miR-34c directly contributes to AD and age-associated memory impairment. The currently accepted view describes miRNAs as important regulators that help to maintain transcriptome homeostasis (Leung & Sharp, 2010). Given that miR-34c is regulated during hippocampal memory formation, it is therefore also likely that aberrant plasticity, at least in part, contributes to pathologically high levels of miR-34c. For example, it has been described that amyloid-beta peptides can induce rather rapid changes in miRNA expression in hippocampal neurons.

Herein SIRT1 has been used as a read out of miR-34c activity in vivo, because SIRT1 was a confirmed target of the miR-34 family in other biological systems and it has been shown that miR-34c also targets Sirt1 mRNA in neurons. Since loss of SIRT1 has been implicated with impaired memory function it is tempting to speculate that miR-34c mediated decrease in hippocampal SIRT1 levels contributes to the observed memory impairment. This is supported by the fact that the treatment of APPPS1-21 mice with miR-34 seed inhibitor reinstated physiological SIRT1 levels. In addition, targeting elevated miR-34c levels in aged mice was able to reinstate learning behavior and physiological SIRT1 levels. In conclusion, the present data suggest that deregulation of the circuit involving miR-34c and SIRT1 contributes to age associated cognitive decline. It is however unlikely that SIRT1 is the sole target by which miR-34c affects memory function and further studies are needed to reveal the precise pathways through which this miRNA controls memory formation.

While high levels of miR-34c in disease models correlate with impaired memory function, miR-34c levels are transiently elevated 3 h after mice are exposed to contextual fear conditioning. Interestingly, the transient increase of miR-34c expression in response to fear conditioning follows the gene expression wave of its predicted targets (see FIG. 2) and supports a feedback regulation model similar to that already shown for this miRNA in other biological contexts. However, it should be noted that molecular mechanisms underlying the observed improvement of memory function after inhibition of miR-34c activity may differ between young wild type mice and disease models. In disease models miR-34c levels are chronically elevated which results in decreased SIRT1 protein levels. Targeting the miR-34 seed in disease models reinstates physiological miR-34c activity and SIRT1 protein levels. Inhibition of miR-34c in young wilted type mice may act through other mechanisms, which may include the regulation of other proteins than SIRT1. Indeed, another experimentally confirmed target of miR-34 family is c-MYC which is also regulated via miR-34c during contextual fear conditioning, suggesting a multidimensional action of miR-34c.

miRNAs control expression levels by degradation of target mRNA, translational repression or both. In the disease models tested here, mRNA levels of Sirt1 remained unchanged despite the significant changes in protein levels. miRNA target protection of Sirt1 mRNA by abnormally high levels of miR-34c also did not affect mRNA levels. Thus, in case of SIRT1, the present data shows that in the biological context of aging and AD, control by miR-34c is mainly realized through translational repression.

The use of virus free methods to target gene expression through miRNAs is a promising approach for gene therapy and has been recently applied to the adult mouse brain. The present study reveals the therapeutic potential of such an approach especially for treatment of dementias. Although this technology has just evolved and many technical challenges and questions remain open, the present data shows that targeting miRNA function in hippocampus with seed inhibitors can have a remarkable effect on the regulation of the respective miRNA targets and cognitive function. Thus, the approach outlined herein are of great importance in treating diseases like Alzheimer characterized by abnormal levels of a miRNAs.

In conclusion, by using massive parallel sequencing miR-34c has been identified as a candidate miRNA implicated with hippocampal and memory function. Furthermore, miR-34c is significantly up-regulated in mouse models for AD and in AD patients. Notably, targeting miR-34c rescues memory function in a mouse model for AD-linked amyloid pathology suggesting that strategies to manipulate miR-34c could open a suitable novel virus-free therapeutic avenue to treat cognitive diseases.

1. A method for preventing or treating memory impairment and/or a neurodegenerative condition, disease or disorder in a subject in need thereof, comprising administering to the subject a therapeutically effective amount of a compound targeting a miRNA or any precursor, or targeting the activity of said miRNA or any precursor thereof.

2. The method according to claim 1 wherein the targeted miRNA is miR-34 or any precursor thereof, or a miRNA sharing the same miRNA seed with miR-34 or any precursor thereof.

3. The method according to claim 1 wherein the targeted miRNA is miR-34c or any precursor thereof.

4. The method according to claim 1 wherein said compound to be administered is an oligonucleotide, in particular, a RNA or DNA oligonucleotide, which may be modified or an artificially synthesized polymer able to interfere between the miRNA and the target and/or reduce the expression of said miRNA or antagonize the activity of said miRNA.

5. The method according to claim 1 wherein said compound is an oligopeptide or a chemically modified oligopeptide, in particular, a protein, an enzyme, an antibody or a fragment of an antibody.

6. The method according to claim 1 wherein said compound is an antisense miRNA compound being an oligomeric compound of 15 to 49 nucleobases in length.

7. The method according to claim 1 wherein the compound has at least 90% sequence complementarity, preferably at least 95% sequence complementarity, to a target region within the miRNA or precursor thereof, like pre-miRNA or pri-miRNA, in particular, has at least 100% sequence complementarity to the miR34 seed sequence.

8. The method according to claim 1 wherein the condition, disease or disorder is a memory impairment condition selected from brain trauma, strokes, meningitis, epilepsy, subjective mild cognitive impairment, mild cognitive impairment.

9. The method according to claim 1 wherein the neurodegenerative condition is a dementia, in particular, Alzheimer's disease HIV-related dementia, frontotemporal dementia, vascular dementia, Lewy body disease, mixed dementias, familial Danish Dementia, familial British Dementia, inclusion body myositis (IBM), or neuronal disorder related to aging processes.

10. A method according to claim 1 wherein the disease or disorder is a neuropsychiatric condition, such as anxiety disorders or depression disorders, in particular, posttraumatic stress disorder, phobia, major depressive disorder, schizophrenia.

11. The method according to claim 1 wherein the oligomeric compound contains at least one modified monomeric subunit, preferably a modified nucleoside or a nucleoside mimetic.

12. The method according to claim 1 wherein administration is effected by the stereotactic injection, intravenous, oral, nasal, intracranial, intramuscular route, or injection into the cerebrospinal fluid.

13. The method according to claim 1 wherein the compound comprises a vehicle for promoting size-specific delivery, in particular, a cholesterol-tag, or other lipid tags or bio functionalized (e.g. antibody-tag) artificial particles, like polybutylcyanoacrylate (PBCA)-stabilized microbubbles, exosomes or nano-or microparticles mediating cell specific drug delivery containing said compound for delivery thereof.

14. A pharmaceutical composition containing a compound targeting miR-34, pre-miR-34 precursor, pri-miR-34 precursor, or a miRNA sharing the same miRNA seed with miR-34, in particular, targeting miR-34c or any precursor thereof together with a pharmaceutically acceptable carrier, diluent or recipient.

15. The pharmaceutical composition according to claim 14 wherein the compound is present in form of naked oligonucleotides or lipid membrane vessels, like liposomes or exosomes, or polybutylcyanoacrylate (PBCA)-stabilized micro- or nanobubbles or micro- or nanoparticles.

16. The pharmaceutical composition according to claim 14 whereas the pharmaceutically acceptable carrier are lipids, polymers, lipopolyamines, in particular, small lipid bodies, e.g. lipid nanoparticles or nanotubes, liposomes, exosomes.

17. A method of inducing memory loss in an individual comprising administering to said individual an effective amount of one or more compounds having the functionality of the small-noncoding miR-34, in particular miR-34c or any precursor thereof or a miRNA sharing the same miRNA seed with miR-34, in particular, administering miRNA-34c or any precursor thereof.

18. The method according to claim 17 wherein memory loss is due to a neuropsychiatric condition involving pathological memories like excessive fear memory as in anxiety disorders or depression disorders.

19. The method according to claim 17 wherein said neuropsychiatric condition is elected from posttraumatic stress disorder, phobia, major depressive disorder, schizophrenia.

20. A method for improving memory functionability in a subject suffering from memory loss, the method comprising reducing the level of miR-34 levels or precursor molecule levels in brain tissue of the subject by administering to the subject a therapeutically effective amount of a compound targeting miR-34 or any precursor thereof, or a miRNA sharing the same miRNA seed with miR-34 or any precursor thereof.

21. A method for diagnosing or predicting memory impairment and/or a neurodegenerative condition, disease or disorder in a subject comprising the steps of
determining the level of expression of miR-34 or any precursor thereof, in particular, miR-34c in a biological sample, like in tissue, blood, liquor or cerebrospinal fluid sample
comparing the level of expression of miR-34 or any precursor thereof, in particular, miR-34c, with a reference sample of a subject not afflicted with or suspected to have memory impairment and/or a neurodegenerative condition, disease or disorder;
predicting or diagnosing memory impairment and/or a neurodegenerative condition, disease or disorder based on the expression level of miR-34 or any precursor thereof, in particular, based on miR-34c expression level in the biological sample, like in tissue, blood, liquor or cerebrospinal fluid.


SEQUENCE LISTING (103 sequences)

SEQ ID NO: 1 - RNA, 23 nt, Homo sapiens
uggcagugucuuagcugguuguu

SEQ ID NO: 2 - RNA, 23 nt, Homo sapiens
uaggcagugucauuagcugauug

SEQ ID NO: 3 - RNA, 23 nt, Homo sapiens
aggcaguguaguuagcugauugc

SEQ ID NO: 4 - RNA, 22 nt, Caenorhabditis briggsae
aggcagugugguuagcugguug

SEQ ID NO: 5 - RNA, 23 nt, Rattus norvegicus
uaggcaguguaauuagcugauug

SEQ ID NO: 6 - RNA, 22 nt, Drosophila pseudoobscura
uggcagugugguuagcugguug

SEQ ID NO: 7 - RNA, 22 nt, Caenorhabditis elegans
aggcagugugguuagcugguug

SEQ ID NO: 8 - RNA, 22 nt, Macaca mulatta
uggcagugucuuagcugguugu

SEQ ID NO: 9 - RNA, 23 nt, Mus musculus
aggcaguguaauuagcugauugu

SEQ ID NO: 10 - RNA, 22 nt, Sanguinus labiatus
uggcagugucuuagcugguugu

SEQ ID NO: 11 - RNA, 22 nt, Pongo pygmaeus
uggcagugucuuagcugguugu

SEQ ID NO: 12 - RNA, 22 nt, Bos taurus
aggcaguguaguuagcugauug

SEQ ID NO: 13 - RNA, 23 nt, Danio rerio
aggcagugcaguuaguugauuac

SEQ ID NO: 14 - RNA, 22 nt, Mus musculus
uggcagugucuuagcugguugu

SEQ ID NO: 15 - DNA, 23 bp, Rattus norvegicus
uggcagugucuuagcugguuguu

SEQ ID NO: 16 - RNA, 22 nt, Bos taurus
aggcaguguaauuagcugauug

SEQ ID NO: 17 - DNA, 22 bp, Drosophila melanogaster
uggcagugugguuagcugguug

SEQ ID NO: 18 - DNA, 22 bp, Gorilla gorilla
uggcagugucuuagcugguugu

SEQ ID NO: 19 - DNA, 23 bp, Monodelphis domestica
uggcagugucuuagcugguuguu

SEQ ID NO: 20 - DNA, 23 bp, Gallus gallus
uggcagugucuuagcugguuguu

SEQ ID NO: 21 - DNA, 22 bp, Ateles geoffroyi
uggcagugucuuagcugguugu

SEQ ID NO: 22 - RNA, 23 nt, Gallus gallus
caggcaguguaguuagcugauug

SEQ ID NO: 23 - DNA, 22 bp, Lagothrix lagotricha
uggcagugucuuagcugguugu

SEQ ID NO: 24 - DNA, 23 bp, Gallus gallus
aggcaguguaguuagcugauugc

SEQ ID NO: 25 - DNA, 23 bp, Xenopus tropicalis
caggcaguguaguuagcugauug

SEQ ID NO: 26 - DNA, 22 bp, Pan paniscus
uggcagugucuuagcugguugu

SEQ ID NO: 27 - DNA, 23 bp, Mus musculus
aggcaguguaguuagcugauugc

SEQ ID NO: 28 - DNA, 22 bp, Danio rerio
uggcagugucuuagcugguugu

SEQ ID NO: 29 - DNA, 23 bp, Xenopus tropicalis
uggcagugucuuagcugguuguu

SEQ ID NO: 30 - DNA, 22 bp, Bombyx mori
uggcagugugguuagcugguug

SEQ ID NO: 31 - DNA, 23 bp, Danio rerio
uaggcaguguuguuagcugauug

SEQ ID NO: 32 - DNA, 23 bp, Rattus norvegicus
aggcaguguaguuagcugauugc

SEQ ID NO: 33 - DNA, 22 bp, Macaca nemestrina
uggcagugucuuagcugguugu

SEQ ID NO: 34 - DNA, 22 bp, Pan troglodytes
uggcagugucuuagcugguugu

SEQ ID NO: 35 - DNA, 110 bp, Homo sapiens
ggccagcugugaguguuucuuuggcagugucuuagcugguuguugugagcaauaguaaggaagcaaucagcaaguauacugcccuagaagugcugcacguuguggggccc

SEQ ID NO: 36 - DNA, 84 bp, Homo sapiens
gugcucgguuuguaggcagugucauuagcugauuguacuguggugguuacaaucacuaacuccacugccaucaaaacaaggcac

SEQ ID NO: 37 - DNA, 77 bp, Homo sapiens
agucuaguuacuaggcaguguaguuagcugauugcuaauaguaccaaucacuaaccacacggccagguaaaaagauu

SEQ ID NO: 38 - DNA, 74 bp, Gallus gallus
agccugguuaccaggcaguguaguuagcugauugccaccaggaccaaucacuaaccacacagccagguaaaaag

SEQ ID NO: 39 - DNA, 67 bp, Xenopus tropicalis
uucaggcaguguaguuagcugauuguguuauaucaaauuugcaaucacuagcuaaacuaccauaaaa

SEQ ID NO: 40 - DNA, 111 bp, Ateles geoffroyi
ggccagcugugaguguuucuuuggcagugucuuagcugguuguugugugcaauagugaaggaagcaaucagcaaguauacugcccuagaagugcugcacguuguggggccc

SEQ ID NO: 41 - DNA, 110 bp, Pan troglodytes; identical to SEQ ID NO: 35

SEQ ID NO: 42 - DNA, 84 bp, Bos taurus
gugcucgguuuguaggcaguguaauuagcugauuguacucucaugcuuacaaucacuaguuccacugccaucaaaacaaggcac

SEQ ID NO: 43 - DNA, 110 bp, Macaca nemestrina
ggccagcugugaguguuucuuuggcagugucuuagcugguuguugugagcaauaguaaggaagcaaucagcaaguauacugcccuagaagugcuacacauuguggggccu

SEQ ID NO: 44 - DNA, 84 bp, Gallus gallus
gugcuugguuugcaggcaguguaguuagcugauuguacccagcgccccacaaucacuaaauucacugccaucaaaacaaggcac

SEQ ID NO: 45 - DNA, 77 bp, Rattus norvegicus
agucuaguuacuaggcaguguaguuagcugauugcuaauaguaccaaucacuaaccacacagccagguaaaaagacu

SEQ ID NO: 46 - DNA, 67 bp, Xenopus tropicalis; identical to SEQ ID NO: 39

SEQ ID NO: 47 - DNA, 96 bp, Xenopus tropicalis
cugugaguguuucuuuggcagugucuuagcugguuguuguggcacguuauagaaguagcaaucagcaaauauacugcccuagaaguucugcacauu

SEQ ID NO: 48 - DNA, 77 bp, Mus musculus; identical to SEQ ID NO: 45

SEQ ID NO: 49 - DNA, 111 bp, Lagothrix lagotricha
ggccagcugugaguguuucuuuggcagugucuuagcugguuguugugagcaauagugaaggaagcaaucagcaaguauacugcccuagaagugcugcacguuguggggccc

SEQ ID NO: 50 - DNA, 98 bp, Bombyx mori
agaaucaggguagaccgcguuggcagugugguuagcugguuguguauggaaaugacaacagccacuaacgacacugcuccugcgugcacccuaaauca

SEQ ID NO: 51 - DNA, 111 bp, Saguinus labiatus
ggccggcugugaguguuucuuuggcagugucuuagcugguuguugugagcaauagugaaggaagcaaucagcaaguauacugcccuagaagugcugcacguuguggggccc

SEQ ID NO: 52 - DNA, 84 bp, Danio rerio
ugcuguguggucaccaggcagugcaguuaguugauuacaauccauaaaguaaucacuaaccucacuaccaggugaaggcuagua

SEQ ID NO: 53 - DNA, 84 bp, Rattus norvegicus
gugcucgguuuguaggcaguguaauuagcugauuguagugcggugcugacaaucacuaacuccacugccaucaaaacaaggcac

SEQ ID NO: 54 - DNA, 111 bp, Monodelphis domestica
ggccagcugugaguguuucuuuggcagugucuuagcugguuguugugaguaauagauaaggaagcaaucagcaaguauacugcccuagaagugcugcacguuguuaggccc

SEQ ID NO: 55 - DNA, 110 bp, Gorilla gorilla; identical to SEQ ID NO: 35

SEQ ID NO: 56 - DNA, 110 bp, Macaca mulatta; identical to SEQ ID NO: 43

SEQ ID NO: 57 - DNA, 85 bp, Danio rerio
gggguuggucuguaggcaguguuguuagcugauuguuucauaugaacuauaaucacuaaccauacugccaacacaacaaccuaca

SEQ ID NO: 58 - DNA, 98 bp, Danio rerio
cugcugugagugguucucuggcagugucuuagcugguuguuguguggagugagaacgaagcaaucagcaaguauacugccgcagaaacucgucaccuu

SEQ ID NO: 59 - DNA, 102 bp, Mus musculus
ccagcugugaguaauucuuuggcagugucuuagcugguuguugugaguauuagcuaaggaagcaaucagcaaguauacugcccuagaagugcugcacauugu

SEQ ID NO: 60 - DNA, 110 bp, Pan paniscus
ggccagcugugaguguuucuuuggcagugucuuagcugguuguugugagcaauaguaaggaagcaaucagcaaguauacugcccuagaagugcugcacguuguggccccc

SEQ ID NO: 61 - DNA, 102 bp, Rattus norvegicus
ccggcugugaguaauucuuuggcagugucuuagcugguuguugugaguauuagcuaaggaagcaaucagcaaguauacugcccuagaagugcugcacguugu

SEQ ID NO: 62 - DNA, 85 bp, Xenopus tropicalis
uguuggguuuucaggcaguguaguuagcugauuguguuaacauaagacuugcaaucacuagcuaaacuaccagcaaaacuaaaca

SEQ ID NO: 63 - DNA, 110 bp, Pongo pygmaeus; identical to SEQ ID NO: 35

SEQ ID NO: 64 - DNA, 85 bp, Xenopus tropicalis; identical to SEQ ID NO: 62

SEQ ID NO: 65 - DNA, 93 bp, Caenorhabditis briggsae
aagcacucauggucgugaggcagugugguuagcugguugcauacacagguugacaacggcuaccuucacugccaccccgaacauguaguccuc

SEQ ID NO: 66 - DNA, 109 bp, Gallus gallus
gccagcugugaguguuucuuuggcagugucuuagcugguuguugugagcaauaguuaaggaagcaaucagcaaguauacugcccuagaagugcuacacauuguugggcc

SEQ ID NO: 67 - DNA, 77 bp, Bos taurus
agucuaguuacuaggcaguguaguuagcugauugcuaauaauaccaaucacuaaccacacggccagguaaaaagauu

SEQ ID NO: 68 - DNA, 100 bp, Drosophila pseudoobscura
aauuggcuaugcgcuuuggcagugugguuagcugguuguguagccaaaauauugccuuugaccauucacagccacuaucuucacugccgccgcgacaagc

SEQ ID NO: 69 - DNA, 99 bp, Drosophila melanogaster
aauuggcuaugcgcuuuggcagugugguuagcugguuguguagccaauuauugccguugacaauucacagccacuaucuucacugccgccgcgacaagc

SEQ ID NO: 70 - DNA, 84 bp, Mus musculus; identical to SEQ ID NO: 53

SEQ ID NO: 71 - DNA, 97 bp, Caenorhabditis elegans
cggacaaugcucgagaggcagugugguuagcugguugcauauuuccuugacaacggcuaccuucacugccaccccgaacaugucguccaucuuugaa

SEQ ID NO: 72 - DNA, 6 bp, Artificial Sequence
  Synthetic oligonucleotide
ggcagg

SEQ ID NO: 73 - protein, 16 aa, Artificial Sequence
  Synthetic peptide sequence
RQIKIWFQNRRMKWKK

SEQ ID NO: 74 - protein, 14 aa, Artificial Sequence
  Synthetic peptide sequence
GRKKRRQRRRPPQC

SEQ ID NO: 75 - protein, 27 aa, Artificial Sequence
  Synthetic peptide signal sequence
GALFLGWLGAAGSTMGAWSQPKKKRKV

SEQ ID NO: 76 - protein, 18 aa, Artificial Sequence
  Synthetic peptide sequence
LLIILRRRIRKQAHAHSK

SEQ ID NO: 77 - protein, 26 aa, Artificial Sequence
  Synthetic peptide sequence
GWTLNSAGYLLKTNLKALAALAKKIL

SEQ ID NO: 78 - protein, 18 aa, Artificial Sequence
  Synthetic peptide sequence
KLALKLALKALKAALKLA

SEQ ID NO: 79 - protein, 9 aa, Artificial Sequence
  Synthetic peptide sequence
RRRRRRRRR

SEQ ID NO: 80 - protein, 31 aa, Artificial Sequence
  Synthetic peptide sequence
SWLSKTAKKLENSAKKRISEGIAIAIQGGPR

SEQ ID NO: 81 - protein, 30 aa, Artificial Sequence
  Synthetic peptide sequence
ACYCRIPACIAGERRYGTCIYQGRLWAFCC

SEQ ID NO: 82 - protein, 36 aa, Artificial Sequence
  Synthetic peptide sequence
DHYNCVSSGGQCLYSACPIFTKIQGTCYRGKAKCCK

SEQ ID NO: 83 - protein, 11 aa, Artificial Sequence
  Synthetic peptide sequence
RKCRIWIRVCR

SEQ ID NO: 84 - protein, 42 aa, Artificial Sequence
  Synthetic peptide sequence
RRRPRPPYLPRPRPPPFFPPRLPPRIPPGFPPRFPPRFPGKR

SEQ ID NO: 85 - protein, 12 aa, Artificial Sequence
  Synthetic peptide sequence
LPWKWPWWPWRR

SEQ ID NO: 86 - protein, 16 aa, Artificial Sequence
  Synthetic peptide sequence
AAVALLPAVLLALLAP

SEQ ID NO: 87 - protein, 11 aa, Artificial Sequence
  Synthetic peptide sequence
AALLPVLLAAP

SEQ ID NO: 88 - protein, 13 aa, Artificial Sequence
  Synthetic peptide sequence
GRKKRRQRRRPPQ

SEQ ID NO: 89 - protein, 16 aa, Artificial Sequence
  Synthetic peptide sequence
RQIKIWFQNRRMKWKK

SEQ ID NO: 90 - DNA, 40 bp, Artificial Sequence
  Synthetic oligonucleotide
agatcttcaccacaaatactgccaagatgtgaatatgcaa

SEQ ID NO: 91 - DNA, 40 bp, Artificial Sequence
  Synthetic oligonucleotide
acccagttaggaccattactgccagaggagaaaagtatta

SEQ ID NO: 92 - DNA, 24 bp, Artificial Sequence
  Synthetic oligonucleotide
cgtggagacatttttaatcaggta

SEQ ID NO: 93 - DNA, 19 bp, Artificial Sequence
  Synthetic oligonucleotide
gcttcatgatggcaagtgg

SEQ ID NO: 94 - DNA, 19 bp, Artificial Sequence
  Synthetic oligonucleotide
tcctcctcagaccgctttt

SEQ ID NO: 95 - DNA, 21 bp, Artificial Sequence
  Synthetic oligonucleotide
cctggttcatcatcgctaatc

SEQ ID NO: 96 - DNA, 25 bp, Artificial Sequence
  Synthetic oligonucleotide
atctcgtatgccgtcttctgcttgt

SEQ ID NO: 97 - DNA, 31 bp, Artificial Sequence
  Synthetic oligonucleotide
gatgctagcacagcttggctgacgatttgct

SEQ ID NO: 98 - DNA, 31 bp, Artificial Sequence
  Synthetic oligonucleotide
ataggcgcgccctatggctctgtcctcacca

SEQ ID NO: 99 - DNA, 55 bp, Artificial Sequence
  Synthetic oligonucleotide
cgcgccaggcagtgtagttagctgattggatccaggcagtgtagttagctgattg

SEQ ID NO: 100 - DNA, 36 bp, Artificial Sequence
  Synthetic oligonucleotide
ataggcgcgccactattgaagctgtccggattcagg

SEQ ID NO: 101 - DNA, 40 bp, Artificial Sequence
  Synthetic oligonucleotide
atagaattccagtcattaaacggtctacaaaacatatgcc

SEQ ID NO: 102 - DNA, 23 bp, Artificial Sequence
  Synthetic oligonucleotide
atcttcaccacaaatactgccaa

SEQ ID NO: 103 - DNA, 20 bp, Artificial Sequence
  Synthetic oligonucleotide
agttaggaccattactgcca